(19) United States
(12) Patent Application Publication
(10) Pub. No.: US 2024/0001155 A1
(43) Pub. Date: Jan. 4, 2024

(54) REVERSAL OF CELLULAR SENESCENCE BY TREATMENT WITH LOW FREQUENCY ULTRASOUND

(71) Applicant: Mechanobiologics, Inc., Galveston, TX (US)

(72) Inventors: Michael Sheetz, Galveston, TX (US); Sanjay Kumar Kureel, Galveston, TX (US); Felix Martin Margadant, Maienfeld (CH)

(73) Assignee: Mechanobiologics, Inc., Galveston, TX (US)

(21) Appl. No.: 18/217,362

(22) Filed: Jun. 30, 2023

Related U.S. Application Data
(60) Provisional application No. 63/357,618, filed on Jun. 30, 2022.

(51) Int. Cl. A61N 7/00; A61K 45/06

(52) U.S. Cl. CPC A61N 7/00; A61K 45/06; A61N 2007/0017

(57) ABSTRACT

Senescent cells are implicated in aspects of age-related decline in health and may contribute to certain diseases. Senescent cells also limit the viability of cells in cell culture. The invention includes methods of using a stressor (e.g., shock waves, pneumatics, hydraulics, magnetic elements, etc.) to reverse senescence. In embodiments, cells are exposed to ultrasound to reverse senescence. Ultrasound can be used to administered in short duration pressure waves to cells to create mechanical stresses. These conditions are safe for normal tissue and do not adversely influence their function. The stressor can rejuvenate senescent cells so that they are phenotypically normal. Additional embodiments include methods of treating senescence-associated diseases and disorders by administering a stressor such as low frequency ultrasound (LFU). The methods described herein can also be used for slowing the aging process and/or reducing signs of aging.

### RELATED APPLICATIONS

This application claims priority to U.S. provisional patent application No. 63/357,618 filed Jun. 30, 2022, the contents of which are incorporated herein by reference.

### FIELD OF THE INVENTION

The invention relates to therapeutics, and more specifically, to methods using a stressor such as ultrasonic radiation to rejuvenate senescent cells and treat senescence associated diseases and disorders.

### BACKGROUND

Aging can be defined as the process of becoming older. In humans, aging represents the accumulation of changes over time and can encompass physical, psychological, and social changes. Advanced age is the greatest risk factor for many chronic diseases. More than 90% of adults aged 65 or older experience at least one chronic disease such as cancer, diabetes, or cardiovascular disease. Aging phenotypes and pathologies, including diverse age-associated diseases and disorders, are causally linked to the accumulation of senescent cell burden with age. Development of therapeutic approaches that slow, stop, or even reverse physiological and pathological changes associated with aging have been considered as an important goal for gerontological research.

Cellular senescence is often defined as a state of permanent cell cycle arrest that was initially defined for cells grown in cell culture. Recently, senescence has been identified both in vitro and in vivo for cells subjected to different forms of stress, and more recently it has also been involved in physiological situations during development.

Senescent cells are characterized by irreversible cell-cycle arrest of proliferation-competent cells, morphological and metabolic changes, altered gene expression, chromatin reorganization, and a unique pro-inflammatory senescence-associated secretory phenotype (SASP). Replicative senescence is activated upon serial passage of cells in culture (or as cells become older in an organism). Senescence can also be induced by different insults such as oncogene activation, irradiation and exposure to chemotherapeutic drugs. Moreover, there are several drugs (e.g., CDK4/CDK6 inhibitors such as Palbociclib) that induce senescence.

Senescence is characterized by a state of permanent cell cycle arrest where cells are metabolically active but stop dividing. The understanding of senescence is important because the accumulation of senescent cells in ageing tissue causes age-related neurodegenerative and cardiovascular pathologies. Further, senescent cells accumulate in most tissues, including the lung, adipose tissue, aorta, and in osteoarthritis joints. There are potentially many benefits from decreasing the accumulation of senescent cells in tissues.

Efforts to develop therapies for senescent related ailments have focused on methods of eliminating senescent cells without affecting non-senescent cells. Recent studies have demonstrated that removal of senescent cells via genetic manipulation in transgenic mouse models can prevent or delay tissue dysfunction, improve age-related pathologies, and extend health span. An inactivated apoptotic pathway in senescent cells enables them to survive many stress signals; however, senolytic agents can kill senescent cells by selectively activating the apoptotic pathway of senescent cells, which reverses some aspects of aging in older mice. These agents are now under consideration for clinical trials to aid older individuals. Several senolytic compounds have shown promising results in mice and human cell culture models. Known compounds include dasatinib and quercetin, piperlongumine and Bcl2-family inhibitors such as ABT263 and ABT737. While these agents have demonstrated some success in selectively targeting senescent cells, they have limitations.

Known senolytics have been shown to have significant side-effects. As a result, they cannot be administered at doses effective to achieve a desired effect. For example, ABT263 (also known as navitoclax) has dose-limiting platelet toxicity which presents the risk of causing thrombocytopenia. Other limitations include (1) Duration of treatment (2) specificity (3) insensitivity to different types of senescence and (4) effects on neighboring healthy cells. Another approach is aimed at “rejuvenating” senescent cells instead of eliminating them. However, there is currently no treatment which can either delay senescence or rejuvenate senescent cells to maintain tissue function.

Therapeutic ultrasound is a type of ultrasonic procedure that uses ultrasound for therapeutic benefit. Ultrasound passes through human tissue where it is the main source of the observed biological effect. There is some evidence that ultrasound is more effective than placebo treatment for treating patients with arthritis pain, a range of musculoskeletal injuries and for promoting tissue healing. Ultrasound has been used to treat various ailments, including ligament sprains, muscle strains, tendonitis, joint inflammation, plantar fasciitis, metatarsalgia, facet irritation, impingement syndrome, bursitis, rheumatoid arthritis, osteoarthritis and scar tissue adhesion.

The Applicants have discovered that external stressors such as ultrasound can “rejuvenate” senescent cells such that normal behavior is restored to the cells. Accordingly, the invention includes methods of returning senescent cells to phenotypically normal cells using a stressor. The methods can be used to maintain cell lines (i.e., in vitro) and therapeutically (i.e., in vivo). Methods of treating senescence-associated diseases or disorders, slowing the aging process and/or reducing signs of aging using ultrasound are also described herein.

### SUMMARY OF THE INVENTION

The inventions described and claimed herein have many attributes and embodiments including, but not limited to, those set forth or described or referenced in this brief summary. The inventions described and claimed herein are not limited to, or by, the features or embodiments identified in this summary, which is included for purposes of illustration only and not restriction.

Methods are described for using a stressor such as low frequency ultrasound (LFU) to rejuvenate senescent cells. The methods can be used to treat senescence associated diseases or disorders. The methods can also be used to mitigate and/or reverse the aging process and reduce signs of aging. The methods can also be used to rejuvenate senescent cells in-vitro cell lines, thereby removing as well.

Embodiments include methods of using a stressor to rejuvenate senescent cells. In aspects, the stressor causes pressure induced mechanical stresses upon target cells. In aspects, the stressor converts senescent cells to phenotypically normal cells. In aspects, the stressor alters mitochondrial and lysosome dynamics and increases mitochondrial fission. In aspects, the stressor is one or more of air pressure, vacuum pressure, hydraulic force, physical compression, physical expansion, electricity, light flashes, microwaves, acoustic shock and ultrasound. In aspects, the stressor is modulated (i.e., low/high intensity) or administered in pulses or bursts (i.e., “stress cycles”).

Embodiments include methods of using a stressor such as low frequency ultrasound (LFU) to rejuvenate senescent cells. In aspects, the stressor is modulated or administered in cycles. In aspects, the stressor causes pressure induced mechanical stresses upon target cells. In aspects, the stressor converts senescent cells to phenotypically normal cells. In aspects, the LFU alters mitochondrial and lysosome dynamics and increases mitochondrial fission. In aspects, the stressor is high frequency ultrasound (i.e., 20-50 MHz).

In embodiments, the stressor is ultrasound that is (a) low frequency ultrasound, (b) low intensity low frequency ultrasound or (c) low intensity pulsed ultrasound. In aspects, the ultrasound is administered to a subject at a “full body ultrasound spa.” In aspects, the LFU is administered in a range from about 20 kHz up to 200 kHz. In aspects, the LFU is administered in pulses (e.g., 1.5 second on followed by 1.5 seconds off).

In embodiments, the stressor is ultrasound that is high intensity pulsed ultrasound. In aspects, the ultrasound is administered to a subject at a “full body ultrasound spa.” In aspects, the ultrasound is administered in a range from about 20 MHz up to 50 MHz or more. In aspects, the ultrasound is administered in pulses (e.g., 1.5 second on followed by 1.5 seconds off).

In embodiments, methods of LFU treatment maintain use sound levels low enough to avoid injury to cells/tissue. In aspects, membranes and other cell organelles experience stress but have no long-term damage (e.g., similar to exercise-induced stress).

In embodiments, senescent cells can be identified with one or more markers. In aspects, senescent cells have reduced β-gal activity, reduced size, increased growth rate, improved ability to replicate, and/or reduced secretion of pro-inflammatory factors (i.e., SASP) after treatment with LFU. In aspects, restoration of normal behavior correlates with a decrease in mitochondrial length and a decrease in lysosomal volume but not with a change in microtubule morphology.

In embodiments, LFU is administered at low to intermediate power levels to induce stress in targeted cells. In aspects, the LFU is administered at medium power levels. In aspects, the LFU is administered by modulating ultrasound pressure (e.g., by modulating the LFU with 1.5 second on and off for 1.5 seconds). The modulation can be repeated for a treatment duration from two minutes to two hours.

Embodiments also include methods of treating senescent cells with a stressor such as ultrasound wave to treat senescence associated diseases and disorders.

Embodiments also include methods of treating cells with a stressor such as ultrasound wave to slow/prevent aging and/or to treat senescence associated diseases and disorders.

Embodiments also include methods of treating senescent cells with a stressor such as ultrasound wave to rejuvenate and/or maintain cells. In aspects, the cells are cultured cells. In other aspects, the cells are cells in an organ or tissue.

Embodiments include methods of treating cells in vitro with a stressor such as ultrasound and using the media or supernatant (i.e., ultrasound induced normal cell secretions or “USINCS”) to rejuvenate and/or maintain cells. In an embodiment, a method of reversing cellular senescence in senescent cells includes steps of (a) administering a stressor such as low frequency ultrasound (LFU) to cultured cells, (b) removing the supernatant from the LFU-treated cultured cells and (c) treating senescent cells with the supernatant.

Embodiments include methods of slowing the aging process and/or reducing signs of aging in a subject. The methods can include administering a therapeutic amount of a stressor such as low frequency ultrasound (LFU).

Embodiments also include methods of reversing senescence in cells. The methods can include administering a therapeutic amount of a stressor such as low frequency ultrasonic (LFU) radiation to the cells. The cells can be cells in culture or cells of a tissue.

Embodiments also include methods of using a stressor such as LFU to benefit tissues in aging organisms. In aspects, phenotypically healthy cells are treated with LFU when there is an expectation that a mixture of senescent and normal cells are present.

In some embodiments, ultrasound is administered using a catheter. The catheter can conduct ultrasonic waves inside tissue. In aspects, rods are used to conduct ultrasonic waves sound throughout a tissue.

In embodiments, the methods include positioning an ultrasound catheter at a treatment site such as a tissue with senescent cells present. In some embodiments, the method can also include administration of a medicament (e.g., by infusion of a from the ultrasound catheter) into a treatment sight. The medicament can be a senolytic agent.

Embodiments include methods of treating senescence-associated disease or disorder, slowing the aging process and/or reducing signs of aging. The method can include selectively targeting senescent cells for rejuvenation. In aspects, the methods can be used to treat diabetes or pre-diabetes.

Embodiments include methods of rejuvenating senescent cells, treating senescence-associated disease or disorder, slowing the aging process and/or reducing signs of aging. The method can include selectively targeting senescent cells for rejuvenation. In aspects, one or more biomarkers can be used to identify senescent cells.

Embodiments also include methods slowing the aging process and/or reduce signs of aging in a subject using a stressor such as ultrasound.

Embodiments include methods of treating humans and animals with a stressor such as ultrasound to improve physical and/or athletic performance.

Embodiments include methods of using a stressor such as ultrasound to decrease mitochondrial length in senescent cells.

Embodiments include methods of using a stressor such as ultrasound to increase lysosome numbers in senescent cells.

Embodiments include methods of using a combination of exercise and a stressor such as ultrasound to treat organisms to improve physical performance.

Embodiments include methods of using a combination of Rho kinase inhibitor and a stressor such as ultrasound to reverse senescence.

Embodiments include methods of using a stressor such as ultrasound for slowing, stopping, and reversing neurodegeneration.

In embodiments, ultrasound is administered to a subject in a full body ultrasound spa.

In embodiments, the ultrasound treatment reduces senescent cell burden in an individual.

In embodiments, pulsed ultrasound (a) improves wound healing, (b) improves bone/tissue healing, (c) improves recovery from injury and/or (d) improves perfusion to tissue. The treatment can be used following an injury or surgery.

Embodiments include a method of treating an ailment associated with an elevated senescent cell burden, the method comprising: (a) identifying an elevated senescent cell burden in a tissue and (b) administering a stress to the tissue, thereby reducing the number of senescent cells (i.e., reducing the senescent cell burden) in the tissue. The stress can be induced by pulsed LFU. In aspects, senescent cells are identified by increased levels of biomarkers, cell morphology, decreased activity, etc.

Embodiments include methods for using a stressor such as ultrasound on donor organs and/or organ recipients before, during, and after the transplantation process to increase the chance of transplantation success and to promote healing.

### BRIEF DESCRIPTION OF THE DRAWINGS

The accompanying drawings illustrate aspects of the present invention. In such drawings:

FIG. 1A shows a series of Brightfield images of senescent cells induced by H₂O₂, sodium butyrate (SB), doxorubicin (Dox), and bleomycin sulphate (BS).

FIG. 1B is a bar graph of the quantification of proliferation (i.e., growth rate) in terms of fold change comparing control cells to senescent cells induced by H₂O₂, sodium butyrate (SB), doxorubicin (DOX) and bleomycin Sulphate (BS).

FIG. 1C is a bar graph comparing the spread area of normal and senescent cells.

FIG. 1D is a bar graph comparing conditioned medium collected from the various senescent cells (SASP) inhibits the growth of normal proliferating cells compared to the control.

FIG. 1E is a pair of images comparing control (i.e., proliferating) cells and senescent cells (SCs) (scale bar=30 μm).

FIG. 1F is a bar graph comparing the cell volume of control and senescent cells (SCs).

FIG. 1G is a pair of images comparing beta-galactosidase staining of control and senescent cells.

FIG. 1H is a bar graph comparing the percentage of beta-galactosidase positive control, H₂O₂ induced senescent cells and sodium butyrate (SB) induced senescent cells.

FIG. 2A is a schematic of senescent cells reversal experiment. Sodium butyrate (SB) induced senescent cells were passaged or treated with ultrasound (US) and passed from P₀ to P₃ every 48 hours.

FIG. 2B is a line graph showing growth rate of ultrasound treated senescent cells (USC), control, and senescent cells.

FIG. 2C is a bar graph showing cell spread area of passaged, ultrasound-treated senescent cells (USC), senescent cells (SCs) and non-senescent cells (NSCs).

FIG. 2D is a bar graph showing ultrasound treatment of senescent cells decreased the number (i.e., percentage) of beta galactosidase positive cells.

FIG. 2E is a pair of images comparing control and ultrasound treated senescent cells stained for P21a.

FIG. 2F is a bar graph showing the fluorescence intensity of p21 stained cells.

FIG. 2G is a pair of images showing control and ultrasound treated senescent cells stained for EDU.

FIG. 2H is a bar graph showing the percentage of EDU positive control and ultrasound treated senescent cells (USC).

FIG. 3A is a schematic of the following experiment: Senescent cells (SCs) were cultured in growth medium for 24 hours. Supernatant was collected before (S0) and 24 hours after ultrasonication treatment (S24). Supernatants S0 and S24 were used to culture non-senescent control cells.

FIG. 3B shows three representative images of cells after 48 hours of incubation in supernatants (control, S0 and S24).

FIG. 3C is a bar graph showing the fold change of cells (i.e., growth rate) after 48 hours in control, S0 or S24 supernatants.

FIG. 3D is a bar graph showing the spread area of cells after 48 hours in control, S0 or S24 supernatants.

FIG. 3E is a schematic diagram of an experiment in which cells were treated with US four times in the same media and the supernatant was collected (USS) for incubation with senescent cells for 48 hours.

FIG. 3F is a pair of Brightfield images illustrating changes in morphology of senescent cells (SCs) after 48 hours in response to supernatant from ultrasound-treated senescent cells (USS). Senescent cells in normal growth medium were used as controls.

FIG. 3G is a bar graph showing the fold change in cell number (i.e., growth rate) between control and ultrasound-treated senescent cells (USS).

FIG. 3H is a bar graph showing the spread area of control and ultrasound-treated senescent cells (USS).

FIG. 4A is a set of six immunofluorescent images showing mitochondrial morphology and lysosome fluorescence in senescent cells stained with mitotracker and lysosome tracker (scale bar=10 μm).

FIG. 4B is a bar graph comparing mitochondrial length of control and ultrasound-treated senescent cells (USCs).

FIG. 4C is a bar graph comparing the ration of intensities of lysosomal to mitochondrial staining between control and ultrasound-treated senescent cells (USCs).

FIG. 4D is a set of images showing J aggregates, Mito monomers, and J aggregates and Mito monomers merged in control cells and ultrasound-treated senescent cells (USCs).

FIG. 4E is a bar graph comparing the Monomer to J aggregate ratio of control and ultrasound-treated senescent cells (USCs).

FIG. 4F is a pair of fluorescence images of microtubules in senescent cells before and after ultrasound treatment (scale bar=10 μm).

FIG. 4G is a bar graph comparing the intensity microtubules in cells before ultrasound (US) treatment and after treatment.

FIG. 5A is a set of four phase contrast images of P13 and P22 control and US treated cells. Control HFF cells were passed every 48 hours from P13 to P24 passage, while ultrasound rejuvenated cells were treated with ultrasound with each other passage.

FIG. 5B is a bar graph comparing the spread area (i.e., growth rate) of control and ultrasound-treated senescent cells (USC).

FIG. 5C is a line graph comparing the cumulative population doubling of ultrasound treated senescent cells (USCs) and control cells as passage number increases.

FIG. 5D is a pair of images comparing the senescence of control and ultrasound treated senescent cells (USCs) at P22 or P24 checked by beta galactosidase staining.

FIG. 5E is a bar graph comparing the percentage of betagel positive cells in control and ultrasound treated senescent cells (USCs).

FIG. 6A is a diagram of the treatment protocol for the mice in subjected to (a) ultrasound, (b) exercise and (c) ultrasound plus exercise.

FIG. 6B is a bar graph showing results of grip tests plotted as the average of the durations.

FIG. 6C is a bar graph showing of results of the inverted cling test.

FIG. 6D is a bar graph showing results of the treadmill tests.

FIG. 6E is a bar graph of results of the rotarod tests.

FIG. 7A is a schematic diagram of an experiment of Resveratrol and EX-527 treatment, followed by ultrasound treatment and medium change.

FIG. 7B is a bar graph showing the change in cell number of untreated control HFF cells in presence of drug.

FIG. 7C is a bar graph that shows the change in cell number of treated P19 cells in the presence of drug.

FIG. 7D is a set of images comparing the immunofluorescence staining of Sirt1 in untreated and US treated HFF cells.

FIG. 7E is bar graph comparing the sirt1 expression by intensity ratio of sirt1 and DAPI in control cells, ultrasound treated cells, EX-527 treated cells, EX-527+US treated cells, resveratrol treated cells and resveratrol+USS treated cells.

FIG. 8A is a bar graph that compares the physical activity (i.e., number of wheel rotations) of control mice (sham) with mice treated with low frequency ultrasound (LFU) for 5 hours, 7.5 hours, 15 hours, 25 hours and 30 hours.

FIG. 8B is a bar graph that compares the endurance time of control mice with two groups of mice treated with low frequency ultrasound (LFU).

FIG. 8C is a bar graph that compares the physical activity (i.e., number of wheel rotations) of control mice with two groups of mice treated with low frequency ultrasound (LFU).

FIG. 8D is a bar graph that compares the lean mass of control mice with two groups of mice treated with low frequency ultrasound (LFU).

FIG. 8E is a graph that compares the survival rate of control mice with two groups of mice treated with low frequency ultrasound (LFU).

FIG. 9A is a bar graph that compares the physical activity (i.e., endurance time on treadmill) of control mice with mice treated with low frequency ultrasound (LFU) for 1 and 6 months.

FIG. 9B is a bar graph that compares the activity level (i.e., turns on an activity wheel) of control mice with mice treated with low frequency ultrasound (LFU).

FIG. 9C is a bar graph that compares the body composition (i.e., percent lean mass) of control mice with mice treated with low frequency ultrasound (LFU).

FIG. 9D is a graph that compares the survival rate of control mice with mice treated with low frequency ultrasound (LFU).

FIG. 9E is a photograph of a “control” mouse that received thirty-minute baths without ultrasound (LFU) treatment.

FIG. 9F is a photograph of a mouse that received two daily treatments of ultrasound (LFU) for six months.

FIG. 10A is a schematic representation of an experiment demonstrating that high-fat, high-sucrose (HFHS) diet increases weight and glucose levels in old mice. The effects can be counter-acted by treating the mice with low frequency ultrasound (LFU).

FIG. 10B is a bar graph that compares blood insulin levels of control mice with mice fed a high-fat, high-sucrose (HFHS) diet. The HFHS mice treated with low frequency ultrasound (LFU) had near normal levels of blood insulin.

FIG. 10C is a bar graph that compares the integrated insulin (i.e., area under the curve) of control mice with mice fed a high-fat, high-sucrose (HFHS) diet. The HFHS mice treated with low frequency ultrasound (LFU) had a higher AUC.

### DEFINITIONS

Reference in this specification to “one embodiment/aspect” or “an embodiment/aspect” means that a particular feature, structure, or characteristic described in connection with the embodiment/aspect is included in at least one embodiment/aspect of the disclosure. The use of the phrase “in one embodiment/aspect” or “in another embodiment/aspect” in various places in the specification are not necessarily all referring to the same embodiment/aspect, nor are separate or alternative embodiments/aspects mutually exclusive of other embodiments/aspects. Moreover, various features are described which may be exhibited by some embodiments/aspects and not by others. Similarly, various requirements are described which may be requirements for some embodiments/aspects but not other embodiments/aspects. Embodiment and aspect can be in certain instances be used interchangeably.

The terms used in this specification generally have their ordinary meanings in the art, within the context of the disclosure, and in the specific context where each term is used. Certain terms that are used to describe the disclosure are discussed below, or elsewhere in the specification, to provide additional guidance to the practitioner regarding the description of the disclosure. It will be appreciated that the same thing can be said in more than one way.

Consequently, alternative language and synonyms may be used for any one or more of the terms discussed herein. Nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only and is not intended to further limit the scope and meaning of the disclosure or of any exemplified term. Likewise, the disclosure is not limited to various embodiments given in this specification.

Without intent to further limit the scope of the disclosure, examples of instruments, apparatus, methods and their related results according to the embodiments of the present disclosure are given below. Note that titles or subtitles may be used in the examples for convenience of a reader, which in no way should limit the scope of the disclosure. Unless otherwise defined, all technical and scientific terms used herein have the same meaning as commonly understood by one of ordinary skill in the art to which this disclosure pertains. In the case of conflict, the present document, including definitions, will control.

The term “senescence” refers to gradual deterioration of functional characteristics in living organisms. Cellular senescence is often defined as a stress-induced, durable cell cycle arrest of previously replication-competent cells. The effects of senescent cells can be thought of as beneficial or detrimental with regard to host physiology and disease, although in some contexts, senescent cells affect a disease state in a complex manner both promoting and opposing certain conditions.

As described herein, a senescent cell can be, for example, a senescent fibroblast, a senescent pre-adipocyte, a senescent epithelial cell, a senescent chondrocyte, a senescent neuron, a senescent smooth muscle cell, a senescent mesenchymal cell, a senescent macrophage or a senescent endothelial cell.

The term “senescence associated secretary phenotype” or “SASP” refers to a phenotype associated with senescent cells wherein those cells secrete high levels of inflammatory cytokines, immune modulators, growth factors, and proteases. SASP can also include exosomes and ectosomes containing enzymes, microRNA, DNA fragments, chemokines, and other bioactive factors. Initially, SASP is immunosuppressive (characterized by TGF-β1 and TGF-β3) and profibrotic, but progresses to become proinflammatory (characterized by IL-1β, IL-6 and IL-8) and fibrolytic. SASP is the primary cause of the detrimental effects of senescent cells.

The term “senescence associated disease or disorder” refers to an ailment that is associated with age and can include, for example, atherosclerosis, osteoarthritis, osteoporosis, hypertension, arthritis, cataracts, cancer, Alzheimer's disease, chronic obstructive pulmonary disease (COPD) and idiopathic pulmonary fibrosis. Other ailments (including age-related conditions) associated with age or senescence include hair graying, sarcopenia, adiposity, neurogenesis, fibrosis and glaucoma.

Still other ailments associated with age or senescence include cardiovascular disease (e.g., atherosclerosis, angina, arrhythmia, cardiomyopathy, congestive heart failure, coronary artery disease, carotid artery disease, endocarditis, coronary thrombosis, myocardial infarction, hypertension, aortic aneurysm, cardiac diastolic dysfunction, hypercholesterolemia, hyperlipidemia, mitral valve prolapsed, peripheral vascular disease, cardiac stress resistance, cardiac fibrosis, brain aneurysm, and stroke). A senescence-associated disease or disorder can also be an inflammatory or autoimmune disease or disorder (e.g., osteoarthritis, osteoporosis, oral mucositis, inflammatory bowel disease or kyphosis). A senescence-associated disease or disorder can also be a neurodegenerative disease (e.g., Alzheimer's disease, Parkinson's disease, Huntington's disease, dementia, mild cognitive impairment or motor neuron dysfunction). A senescence-associated disease or disorder can also be a metabolic disease (e.g., diabetes, diabetic ulcer, metabolic syndrome or obesity). A senescence-associated disease or disorder can also be a pulmonary disease (e.g., pulmonary fibrosis, chronic obstructive pulmonary disease, asthma, cystic fibrosis, emphysema, bronchiectasis or age-related loss of pulmonary function). A senescence-associated disease or disorder can also be an eye disease or disorder (e.g., macular degeneration, glaucoma, cataracts, presbyopia or vision loss). A senescence-associated disease or disorder is an age-related disorder can also be renal disease, renal failure, frailty, hearing loss, muscle fatigue, skin conditions, skin wound healing, liver fibrosis, pancreatic fibrosis, oral submucosa fibrosis or sarcopenia. A senescence-associated disease or disorder can also be a dermatological disease or disorder (e.g., eczema, psoriasis, hyperpigmentation, nevi, rashes, atopic dermatitis, urticaria, diseases or disorders related to photosensitivity or photoaging).

The term “senolytic” or “senolytic agent” refers to a therapeutic such as a small molecule that can selectively or preferentially induce death of senescent cells. A senolytic agent may kill senescent cells by inducing (i.e., activating, stimulating or removing inhibition of) an apoptotic pathway that leads to cell death. Senolytic agents may be useful for treatment of senescence-associated diseases or disorders. For example, the drugs dasatinib, quercetin, fisetin and navitoclax have potential senolytic activities.

The term “rho-kinase inhibitor,” “rho-associated protein kinase inhibitor” or “ROCK inhibitor” refers to a series of compounds that target rho kinase (ROCK) and inhibit the ROCK pathway. ROCK has multiple functions, including regulation of smooth muscle cell contraction, cell migration, and maintenance of cell viability and morphology, in part by regulating stress fibers and focal adhesions. Particularly, ROCK inhibitor is used for cell culture practice, in part to limit cellular death and limited dedifferentiation, and therefore widely adopted for induced pluripotent stem cells (iPSC) and embryonic stem cell cultures, although studies have shown mixed results for other cell types.

The term “passage” or “passage number” refers to the transfer or subculture of cells from one culture vessel to another. The degree of subculturing a cell line has undergone is often expressed as passage number, which can generally be thought of as the number of times cells have been transferred from vessel-to-vessel. Studies indicate that passage number can affect a cell line's characteristics over time. The passage number can be abbreviated as “P.”

The term “senescence-associated ß-galactosidase,” “SA-β-gal” or “SABG” is a hypothetical hydrolase enzyme that catalyzes the hydrolysis of β-galactosidase into monosaccharides only in senescent cells. Senescence-associated beta-galactosidase, along with p16lnk4A, can be used as a biomarker of cellular senescence.

The term “stressor” refers to a chemical or biological agent, environmental condition, external stimulus or an event seen as causing stress to a cell or organism. Stresses to cells and organisms originate from a variety of sources, including mechanical injury, pathogens, toxic compounds in the environment, naturally occurring agents that damage cells such as ultraviolet light, nutrient or oxygen deprivation, etc. Cells can respond to stress in various ways ranging from the activation of survival pathways to the initiation of cell death that eventually eliminates damaged cells. Whether cells mount a protective or destructive stress response depends to a large extent on the nature and duration of the stress as well as the cell type. For example, protective responses such as the heat shock response or the unfolded protein response mediate an increase in chaperone protein activity which enhances the protein folding capacity of the cell, thus counteracting the stress and promoting cell survival.

The term “cellular stress response” refers to a range of molecular changes that cells undergo in response to environmental stressors (e.g., extreme temperature, exposure to toxins, mechanical damage, etc.). The various processes involved in cellular stress responses serve the adaptive purpose of protecting a cell against unfavorable environmental conditions, both through short term mechanisms that minimize acute damage to the cell's overall integrity, and through longer term mechanisms which provide the cell a measure of resiliency against similar adverse conditions.

The term “ultrasound imaging” refers to a versatile modality frequently used in clinical medicine to provide images in real time. Ultrasound used typically in clinical settings has frequencies between 2 and 12 MHz. Lower frequencies produce greater resolution but are limited in depth penetration; higher frequencies produce greater resolution, but depth of penetration is limited. Acoustic energy in the form of ultrasonic sound waves is generated from a transducer as a mechanical vibration. These vibrations are transmitted into the tissues being examined and propagate in the form of an ultrasonic wave that passes easily through fluids and soft tissues. When the wave encounters an interface or a border between two tissues that conduct sound differently, some of the sound waves are reflected back to the transducer, creating an echo. Reflected waves captured by the transducer are amplified electronically and displayed on a monitor.

The term “ultrasonic radiation” or “ultrasonic energy” refers to sound waves with frequencies higher than the upper audible limit of human hearing. For the purposes of this application, “Ultrasonic Radiation” is specifically defined as sound waves with frequencies from 10 kHz up to 15 gigahertz. Exemplary embodiments employ low frequency ultrasound (LFU) energy with a frequency in a range from about 20 kHz to about 500 kHz, an intensity of less than 3 watts/cm².

Medical applications that use ultrasonic energy are known in the art. Conventional uses have been directed at medical imaging. More recently, the use of high intensity focused ultrasound (HIFU) for therapeutic purposes, as opposed to imaging, has been used. HIFU therapy employs ultrasound transducers that can deliver 1,000-10,000 W/cm² at a focal spot, in contrast to diagnostic ultrasound where intensity levels are usually below 0.1 W/cm². A portion of the mechanical energy from these high intensity sound waves is converted at the targeted location to thermal energy. Conventional therapeutic uses of HIFU have generally been directed at destroying or ejecting undesired portions of tissues, to necrose tumors, coagulate bleeding, and address urological and gynecological disorders.

The term “high-frequency ultrasound” or “HFU” generally refers to sound waves greater than 20 MHz.

“Low Frequency Ultrasound” or “LFU” generally refers to sound waves greater than 20 kHz up to and including 200 kHz.

“Low intensity low frequency ultrasound” or “LILFU” refers to low-frequency (0.44-0.67 MHz) with a pulse average intensity (IPA) of 0.3-8 W/cm² and a temporal average intensity (ITA) of 5-50 mW/cm².

“Low intensity pulsed ultrasound” or “LIPU” refers to a technology that can be used for therapeutic purposes. For example, LIPU can be administered at 1.5 MHz frequency pulses, with a pulse width of 200 μs, repeated at 1 kHz, at a spatial average and temporal average intensity of 30 mW/cm.

The term “absorbent material” refers to a material having capacity or tendency to absorb energy from transmitted ultrasound. For example, pyramid-shaped structures that contain a gas or liquid do not allow transmission/reflection of sound waves. Any matter or structured assembly of materials that attenuates ultrasound—in contrast to scattering/dispersing ultrasound—and turns its energy content into heat energy, is considered an “absorbent material” for this purpose.

The term “full body ultrasound spa” refers to a method of using immersion mechanotherapy. The method can include using a vessel containing an amount of medium for immersing a subject and administering cycles of ultrasound waves to through the medium for a pre-determined period of time. See, for example, US 2022/0047894A1.

The “rotarod test” or “rotarod performance test” refers to a performance test based on a rotating rod with necessary motor activity to remain on the rod, usually by a rodent. The test measures parameters such as riding time (seconds) or endurance. Some of the functions of the test include evaluating balance, grip strength and motor coordination of the subjects.

The term “treating” or “treatment” refers to one or more of (1) inhibiting the disease, condition or disorder in an individual who is experiencing or displaying the pathology or symptomatology of the disease, condition or disorder (i.e., arresting further development of the pathology and/or symptomatology); and (2) ameliorating the disease condition or disorder in an individual who is experiencing or displaying the pathology or symptomatology of the disease, condition or disorder (i.e., reversing the pathology and/or symptomatology) such as decreasing the severity of disease.

The term “administration” refers to the introduction of an amount of a predetermined substance into a patient by a certain suitable method. The compositions disclosed herein (e.g., senolytic agents) can be administered via any of the common routes, as long as it is able to reach a desired tissue, for example, inhaling, intraperitoneal, intravenous, intramuscular, subcutaneous, intradermal, oral, topical, intranasal, intrapulmonary, or intrarectal administration.

The term “subject” refers to those who are susceptible to an ailment (e.g., a disease related to senescence) or who are suspected of having or diagnosed with the ailment. However, any subject to be treated with the therapeutic methods described herein is included without limitation. For example, a subject can be a human seeking treatment to inhibit/slow/reverse signs and symptoms of aging or improve athletic performance.

The term “athletic performance” refers to the efforts made by an athlete to attain specific performance objectives over a period of time. For example, an improvement in athletic performance can be greater strength and/or greater endurance.

The term “P21,” “p21 Cip1” or “p21Waf1” also known as cyclin-dependent kinase inhibitor 1 or CDK-interacting protein 1, is a cyclin-dependent kinase inhibitor (CKI) that is capable of inhibiting all cyclin/CDK complexes, though is primarily associated with inhibition of CDK2. p21 represents a major target of p53 activity and thus is associated with linking DNA damage to cell cycle arrest.

The term “5-Ethynyl-2′-deoxyuridine” or “EdU” refers to a thymidine analogue which is incorporated into the DNA of dividing cells. EdU is used to assay DNA synthesis in cell culture and detect cells in embryonic, neonatal and adult animals which have undergone DNA synthesis. Whilst at high doses it can be cytotoxic, this molecule is now widely used to track proliferating cells in multiple biological systems.

All numerical designations, e.g., pH, temperature, time, concentration, and molecular weight, including ranges, are to be understood as approximations in accordance with common practice in the art. When used herein, the term “about” or approximately may connote variation (+) or (−) 1%, 5% 10%, 20%, 30%, 40%, or 50% of the stated amount, as appropriate given the context. It is to be understood, although not always explicitly stated, that the methods, frequencies, reagents, etc. described herein are merely exemplary and that equivalents of such are known in the art.

Other technical terms used herein have their ordinary meaning in the art that they are used, as exemplified by a variety of technical dictionaries. The particular values and configurations discussed in these non-limiting examples can be varied and are cited merely to illustrate at least one embodiment and are not intended to limit the scope thereof.

### DETAILED DESCRIPTION

It is to be understood that both the foregoing general description and the following detailed description are exemplary and explanatory and are intended to provide further explanation of the subject technology as claimed. Additional features and advantages of the subject technology are set forth in the description below, and in part will be apparent from the description, or may be learned by practice of the subject technology. The advantages of the subject technology will be realized and attained by the structure particularly pointed out in the written description and claims hereof.

For the majority of isolated primary cells, they undergo the process of senescence and stop dividing after a certain number of population doublings while generally retaining their viability (described as the Hayflick limit). This means that cell cultures must be constantly refreshed with new cells from the established cell line. It is generally accepted that mammalian cells can only divide for a limited number of times. Gene mutations can accumulate during DNA replication, which ultimately leads to abnormal regulation of cell metabolism and physiology that contribute to the aging processes. Additionally, another well-known mechanism related to cell replicative senescence is the inability of normal somatic cells to fully maintain telomere DNA due to lack of telomerase.

Embodiments of the invention include methods of reversing cellular senescence by exposing cells to mechanical stress (e.g., shock waves, pneumatics, hydraulics, magnetic elements, LFU, etc.). Without being bound by theory, the experimental results described herein indicate that stress (e.g., ultrasound irradiation) acts at a subcellular level on organized elements of the cytoplasm that reflect changes induced by senescence. At a molecular level, Sirtuins have been linked to changes with senescence and particularly Sirtuin1 is implicated in senescence. The results also support a role for Sirtuin1 in the LFU reversal of senescence, because a specific inhibitor of Sirtuin1 blocked the rejuvenation of senescent cells by LFU (see below). Further research is needed to understand how ultrasound pressure waves activate Sirtuin1 enzyme function in rejuvenation.

Because the 30 KHz-50 KHz ultrasound range causes minuscule heating effects (i.e., about four orders of magnitude less than typical harmless diagnostic ultrasound), Applicants regard the cyclical stress cycles imposed on the cellular membrane in particular but also the acceleration stresses for all cell organelles (not directly affected by membrane stress) to be responsible for the downstream effects. While LFU is described in detail as a stressor, other types of stressors are also contemplated.

Most alternatives to ultrasound can still induce membrane stress in individuals (e.g., sports or mechanical stimulation). With the exception of shockwaves or light flashes induced shock, they will not cause any notable acceleration. Applicants have identified several sources of mechanical stress that can have therapeutic benefit:
Pneumatic devices that use confined air pressure to compress tissue. They can cycle at, for example, 0.5 Hz to 1 Hz frequency,
Inversely, local vacuum that expands tissue within the vicinity of application. Over and under-pressure can be combined in pneumatic devices,
Hydraulic forces—cyclically adjusted liquid pressure can be used to stress the tissue. The liquid can make contact with the patient or be confined in a bladder or even a hydraulic cylinder,
Cyclical mechanical stress from compression or pulling/expansion. The force can be generated by a motor, electro-magnet, or via a fluidic or pneumatic system.
Induced muscular stress via nerval stimulation or “electrocution,”
Induced shock by fast repeating light flashes or microwaves (generally electromagnetic pulses) that are absorbed by the tissue and induce a local expansion (usually by absorption and thermal expansion),
Agents that enhance the above absorption such as dyes, pigments, ferrites, or magnetic microbeads
Higher harmonics pressure waves. By not using a sinusoidal wave function, the higher frequency effects can be achieved at much lower base frequencies. Say a rectangular wave has very strong contributions at 2× to 7× the base frequency. If operated at 10 KHz, it will still meet our researched frequency domains. If operated between 18 KHz-20 KHz, it will likely work well without being considered ultrasound and without being heard (audibly) by the majority of patients.
Acoustic shocks—this is more a bypass of the LFU domain by coupling very high pressure sound into an atmosphere, usually a lumen of a capsule just above the treatment area, that then couples into the tissue. Without confinement, this is a very inefficient delivery process. But in a capsule coupling can be improved by more than a magnitude. As there is no air flow, this is fundamentally different from the pneumatic approach. When delivered as shock waves, this combines well with higher harmonics.

Accordingly, an aspect of the methods described herein is the delivery of stress/pressure/sound/irradiation in a cyclically modulated manner. In particular, the stress increases and subsides within specific intervals of 1 to 3 seconds. With ultrasound (LFU or HFU) this is usually implemented as bursts with the sound on for 1.5 seconds then off for a similar duration. In order to “secure” this angle, this also works when the signal is merely modulated, (i.e., if the power is high for 1.5 seconds and lower for 1.5 seconds or if the power undulates with a sinusoidal or related modulation form amplitude change). Accordingly, administration of a stress (e.g., ultrasound) can be described as “bursts of stress” or “modulated intensity stress.”

### Low Frequency Ultrasound (LFU)

Therapeutic ultrasound is a type of ultrasonic procedure that uses ultrasound for therapeutic benefit. Physiotherapeutic ultrasound was introduced into clinical practice in the 1950s, with lithotripsy introduced in the 1980s. Others are at various stages in transitioning from research to clinical use: high-intensity focused ultrasound, HIFU, targeted ultrasound drug delivery, trans-dermal ultrasound drug delivery, ultrasound hemostasis, cancer therapy, and ultrasound assisted thrombolysis. These may use focused ultrasound (FUS) or unfocused ultrasound. Relatively high-power ultrasound can break up stony deposits or tissue.

The present invention includes methods of using low frequency ultrasound (LFU) to reverse senescence. LFU is used to produce short duration pressure waves in the cells that create mechanical stresses. In aspects, the ultrasound levels are transmitted at intensities to induce activity without causing injury to cells or tissue. For example, membranes and other cell organelles experience stress that can increase metabolism and healing processes (e.g., similar to moderate exercise induced stress). However, the ultrasound exposure causes no long-term damage. These conditions are safe for normal tissue and do not adversely influence their function. In aspects, LFU rejuvenates senescent cells so that they are phenotypically normal.

To stay below the damage thresholds, the methods described herein can use intensities comparable to conventional ultrasound diagnostic techniques, with lower carrier frequencies and greater overall power. The intensities are considerably than conventional therapeutic methods that use ultrasound for cauterization, thermal damage, cell lysing, or tissue dislocation or ejection. Controlled beam travel, beam geometry, and vat geometry are key to a successful implementation of the methods described herein.

The methods described herein can use stress that is induced by medium power ultrasound waves. Specifically, LFU and stress modulation is achieved by modulating ultrasound pressure. The Applicants have discovered that constant stress has no (or negligible) positive rejuvenation impact. Accordingly, methods of stress modulation necessary for therapeutic benefits are described herein. For example, LFU can be administered in pulses (e.g., 1.5 second on followed by 1.5 seconds off). The treatment can be administered for a total duration from two minutes to two hours.

In embodiments, the modulation is more complex than a pulse (i.e., on and off cycles). For example, power can be increase and/or decreased over the course of a treatment. Further, the treatment can include periods of silence that promote periods of recovery or healing.

In embodiments, ultrasound is transmitted to pass through a target tissue. This is in contrast to conventional diagnostic and therapeutic methods that convey energy to a particular tissue or group of cells. The methods described herein are inefficient in the sense that clean and directed ultrasound is administered at expense of electrical power. The amount of energy absorbed by the tissue is minimal (i.e., about 1%-3% of the sound power produced). Energies absorbed by a single cell can be considerably lower than a nanowatt. Accordingly, tissue heating is negligible and tissue is not damaged.

To cap the ultrasound peak pressure, the methods are configured to avoid build up standing waves and unplanned foci. Beam steering and frequency sweeps (e.g., jitter or staggering) can be used to homogenize the ultrasound fields. These techniques can also prevent regions with low ultrasound intensity. An ultrasound beam can be terminated to prevent build up of standing waves. For example, absorbed materials can be added to the treatment vat that will suppress high order reflections.

Accordingly, embodiments of the invention include ultrasound treatment, including low frequency pulsed ultrasound treatment, that can be used to slow, stop, and reverse cell senescence. Ultrasound treatment produces short duration pressure waves that create mechanical stresses in the cells. These do not adversely influence functions of normal cells.

In some embodiments ultrasound is used on cells in culture. Senescent cells can be returned to phenotypically normal cells using ultrasonic. The methods can be used to maintain cell lines (i.e., in vitro).

In other embodiments ultrasound is used on cells vivo, either through exposure of the whole individual or through more localized treatment. For example, an ultrasound catheter can be directed at a treatment site such as a diseased tissue or a sight of senescent cells. Senescent cells at the treatment site can be returned to phenotypically normal cells using ultrasonic energy. The methods can also include administration of a medicament (e.g., a senolytic) and/or combined with other therapies including exercise.

Thus, embodiments of the invention include methods of treating cellular senescence in cells and organisms, thereby slowing, stopping, or reducing cellular senescence and the signs of cellular senescence and related disorders and diseases using therapeutically effective applications of ultrasonic radiation. Embodiments of the invention include methods of increasing physical performance in organisms using therapeutically effective applications of ultrasonic radiation.

Any suitable route or mode of administration of can be employed for providing the patient with a therapeutically or prophylactically effective dose of ultrasound. Exemplary routes or modes of administration include but are not limited to low frequency ultrasound therapy, mechanical ultrasound therapy, or any type of ultrasonic procedure that uses ultrasound for therapeutic benefit. Embodiments of the invention may use focused ultrasound (FUD) or unfocused ultrasound.

### EXAMPLES

The following non-limiting examples are provided for illustrative purposes only in order to facilitate a more complete understanding of representative embodiments now contemplated. These examples are intended to be a mere subset of all possible contexts in which the components of the formulation may be combined. Thus, these examples should not be construed to limit any of the embodiments described in the present specification, including those pertaining to the type and amounts of components of the formulation and/or methods and uses thereof.

### Methods

Applicants used four different methods to induce cell senescence in vitro to demonstrate the use of LFU treatment for reversing cell senescence. The methods include (a) repeated replications (i.e., serial passage of cells in culture), (b) hydrogen peroxide (H₂O₂) treatment, (c) sodium butyrate (SB) treatment and (d) bleomycin sulfate (BS) treatment.

### Cell Line and Cell Culture

Human foreskin fibroblast (HFF) were purchased from the ATCC. African monkey kidney derived Vero cells were obtained from the M. Garcia-Blanco lab as a gift. All cell lines were cultured as per manufacturer's protocol. Vero cells and HFF cells were in growth medium containing DMEM and 10% FBS. Cells plated at 20-40% confluency were maintained in an incubator at 370° C. and 5% CO₂.

### Senescence Induction and Quantification

Vero cells were treated with the stressors including 200 μM H₂O₂, 4 mM of sodium butyrate (SB) or 25 μM of bleomycin sulphate (BS) and incubated for 36-48 hours. After washing with PBS and then replacing growth medium with fresh medium, cells were incubated for four days to confirm the growth arrest of senescent cells. Human foreskin fibroblast (HFF) cells were serially passaged up to fifteen times (p15) since replication of these cells was dramatically reduced by p15-p17. Four criteria were used to determine if cells were senescent; (1) cell cycle arrest by determining the growth rate, (2) Increase in cell spread area, (3) development of senescence associated secretory phonotype (SASP) in culture medium, and (4) beta-gal staining. Images of cells were captured with an Evos microscope at 10× magnification after treatment and 48 hours post treatment.

To measure growth by the increase in cell number, 15 random images were captured, then the average number of cells were determined, which was divided by the area of one frame to get the cell density (cells/cm²). Then this seeding density was multiplied by the total area of the dish or well to obtain the total number of cells after ultrasound (US) treatment and after 48 hours of incubation. The total number of cells at 48 hours was divided by the total number of cells just after the treatment to determine the growth rate. A ratio was one would be an indication of no growth.

Senescence was detected by the beta-galactosidase senescence staining kit as per manufacturer's protocol. Briefly, sub confluent senescent cells were stained by the SA-beta galactosidase (β-gal) staining solution and incubated overnight at 37° C. The β-gal-stained cells appeared blue and were senescent cells. The percentages of β-gal positive cells were determined by counting the number of blue cells and dividing by the total number of cells. Cell spread area was determined by capturing the mages of cells with a 10× objective using an Evos microscope. Then, ImageJ software was used to calculate spread area by manually encircling the cell periphery of each cell. A minimum of 150 cells was used for the analysis. To determine the SASP activity, the senescent cells were cultured for one to three days and then supernatant was collected from each dish. This supernatant was used to culture normal cells. Development of a senescence phenotype by the normal cells in supernatant medium confirmed that senescent cells were secreting SASP.

### Ultrasound Treatment of Cells

Prior to ultrasound treatment, the plates containing senescent Vero cells or late passage HFF cells were wrapped with parafilm to avoid contamination and water influx into the plate. The samples were placed on the plastic mesh, which was mounted on the water tank with an ultrasound transducer. Water in the tank was degassed and heated to temperature of 35° C. The distance between the sample and transducer was approximately 9-10 centimeters (cm). Efforts were made to ensure that there were no air-bubbles or air-water interfaces between the water and the sample. Output power of transducer was measured at the plate location by hydrophone. Cells were treated with pressure pulses of 6-7 kilopascal in water. using 32.249 kHz frequency ultrasound for 30 minutes. Cells were treated cyclically for 1.5 seconds on and 1.5 seconds off. After ultrasound treatment, cell plates were returned to the incubator for 48 hours to determine the growth of senescent cells.

### Reversal of Senescence

First, senescence was induced in Vero cells using sodium butyrate (SB). Then senescence was confirmed using growth arrest and β-gal staining methods. The senescent cells were treated using ultrasound of optimized parameters (33 kHz frequency and 6-7 kilopascal). These cells were incubated for 48 hours and the growth and morphology of the cells was measured before the trypsinization. This passage was designated “P0.” These cells were then trypsinized, reseeded, and incubated for 48 hours in the P1 passage. This process was repeated to P3 passage. The cells were evaluated in terms of fold change in number for growth, morphology, β-gal, and EDU staining to check the population of senescent cells. Senescent cells without ultrasound treatment were used as control cells. Typically, by passage P3, the senescent cells exhibited the phenotype of normal proliferating cells.

Passage 15-24 HFF cells were treated with ultrasound at the optimized frequency, power and duty cycle. Cell proliferation was determined by counting the number of cells at the time of seeding and 48 hours after ultrasound (US) treatment. Ultrasound treated HFF cells showed higher fold change in growth than the untreated HFF cells. In the case of P24 HFF cells, they were treated with ultrasound and incubated for 96 hours prior to trypsinization, reseeding and incubation for 48 hours. Proliferation and morphology were measured after 48 hours of incubation. P24, US treated HFF cells became smaller in size, and they also showed dramatically greater proliferation than the untreated P24 HFF cells.

### Ultrasound Treatment of Aged Mice

Aged mice (21-24 months old) were treated in a large (4 L) glass beaker and were within a plastic cylinder of 13 cm height and 152 cm in diameter. A plastic mess was placed on top the cylinder that supported the mice and enabled them to rest their four limbs and body in the water. Degassed and 32-35° C. warm water was poured into the beaker. The level of water kept one inch above the plastic mesh so that the half of the body of the mice remained in water. After the mice were placed in the water, intermittent ultrasound of 32.249 kHz and 6-7 kilopascal was administered to the mice for 30 minutes at duty cycles of 1.5 second on and 1.5 second off. Animals in the ultrasound groups were treated at 72-96 hour intervals for one month (10 treatments). During the ultrasound treatment, mouse activity was carefully observed along with their adaptation to the system. After treatment, the animals were placed in separate cages with tissue paper to dry the animals and then they were returned to their home cage. Control mice were placed in the same water bath for 30 minutes without ultrasound treatment. In the ultrasound treatment procedure, animals were in direct contact with the water. Animals were in water so that ultrasound was attenuated and reflected dramatically at air-water interfaces.

### Physical Assessment of the Mice

For assessment of the effect of ultrasound (US) treatment on physical performance of the mice, six groups of old mice were used: (1) sham, (2) ultrasound treatment, (3) exercise, (4) rapamycin, (5) exercise plus ultrasound and (6) ultrasound treatment plus rapamycin. Each group included four males and four females. In the case of the rapamycin-treated animals, the C57BL/6J mice were fed with encapsulated rapamycin and monitored daily for a month. Animals of ultrasound groups were treated every 72-96 hours for a month. Animals in the exercise group were trained three times per week on a treadmill for 25 minutes in each exercise training session. Prior to starting the experiment, the physical functions and health condition of the mice were assessed (referred to as “pre-assessment”). After one month of ultrasound treatment and exercise sessions, the physical performance and health conditions of the animals were assessed again (referred to as “post-assessment”). Physical performance was determined by the functional assessment tests including grip test, rotarod, treadmill, and inverted cling tests.

Grip test: Grip strength of mice was measured by a grip strength meter in Newtons. The animal was held by the tail gently and placed so that its paws grabbed the bar. The force needed to pull the bar from the animal was the grip strength. A maximum of five trials for the forelimbs were performed in one session.

Rotarod: A rotarod device allowed us to quantify overall neuromuscular function: endurance, balance, and coordination. The whole procedure involves two training days and one day for testing. During the training days, the animals were acclimating to the device. On the test day, three trials were conducted per session with resting periods of 10 minutes in between trials. The outcome measurement was the duration (time) of the animal on the rod.

Inverted Cling: This grip test was useful to quantify muscle strength and endurance by measuring how long the mouse held onto the grid while inverted. Each animal was tested two times with a resting break of 10 minutes between trials. A minimum of 10 seconds holding was required for test validation to exclude a slip. Three trials were conducted in the first test.

Treadmill: The mice were tested for the maximum power output/maximum gait speed and endurance (to exhaustion) by running on a treadmill. The outcome measurement was the running duration. During the training session, the mice were familiarized with the device, first running at a constant speed, later increasing the speed progressively one unit in every 20 seconds. The mice were allowed to rest 10 minutes between trials. Three electric shocks of 0.4 mA ended the trial and animals were given three trials. During the test session, the speed was increased, and the mouse was allowed to run as long as they could before getting three shocks.

Exercise training sessions: Animals of Exercise and Exercise+ultrasound groups were exercised on a treadmill for 25 minutes in every 48 hours, three times in a week for one month. Every exercise session was preceded by 10 minute warm up at 6 cm/s, followed by 10 minute training at 8 cm/s, and 5 min cool down at 6 cm/s. Training was progressive and started at 8 cm/s in the first week but increased to 11 cm/s in week four. The mice were encouraged to run on treadmill with a light electric shock when they stopped running.

There were twelve exercise training sessions and 10 ultrasound treatments in the one-month experimental period.

### Mitochondrial Morphology

Ultrasound treated cells were incubated with Mitotracker (Invitrogen) in 100 μM at 37° C. for 30 min. Then, Images were captured in the Confocal microscope of 15 randomized fields per sample for quantification. AR and form factors were determined using the formula major axis/minor axis and Perimeter 2/(4π× surface area).

### Immunofluorescence Staining

Cells were seeded on 27 mm glass bottom dish (Ibidi), after ultrasound irradiation, cells were washed twice with PBS. Then, the cells were fixed with 4% paraformaldehyde for 10 minutes and permeabilized with 0.2% Triton-X-100 for 5 minutes. The cells were washed with PBS thrice and incubated with 3% blocking buffer for one hour. Anti-LAMP primary antibody (1:400) was incubated with sample at 4 C overnight followed by washing and incubation with Alexa Flour 488 secondary antibody (1:1000) at room temperature for 4 hours. Images were captured with a confocal microscope.

### Mitochondrial ROS

MitoSOX (Invitrogen) red was used to measure the mitochondrial reactive oxygen species production. Briefly, 5 μl

of MitoSOX was added to the growth medium for 10 minutes at 37° C. Fluorescence images were captured in a confocal microscope (Olympus). ROS level was determined by the intensity measurement.

### Live and Dead Cell Assay

Live cells were detected using Calcine AM (Sigma Aldrich) as per manufacturer's instructions. Briefly, adherent cells were treated with Calcein AM in Opti-MEM medium and incubated for 30 minutes. Apoptotic/Dead cells were identified using Annexin V-FITC/Propidium iodide (PI) (Sigma Aldrich) as per the manufacturer's instructions. Live and dead assay was performed immediately after the ultrasound irradiation and post 24 hours of treatment.

### Calcium Release Assay

Release of calcium was measured using calcium dye (4 mM of Cal-520 AM, AAT Bioquest) as per manufacturer's protocol. Semiconfluent cells were incubated in calcium dye for 30 minutes prior to ultrasound irradiation.

### Statistical Analysis

All results are shown as mean±s. d. and paired t-test, two tails used for two groups. GraphPad Prism 8.4.3. was used for making graph and statistical analysis. *P values<0.05, **p values<0.002, ***p values<0.001, and non-significant (ns) p-value >0.05 were used, shown in figures.

### Example 1

### Ultrasound Reverses Cell Senescence

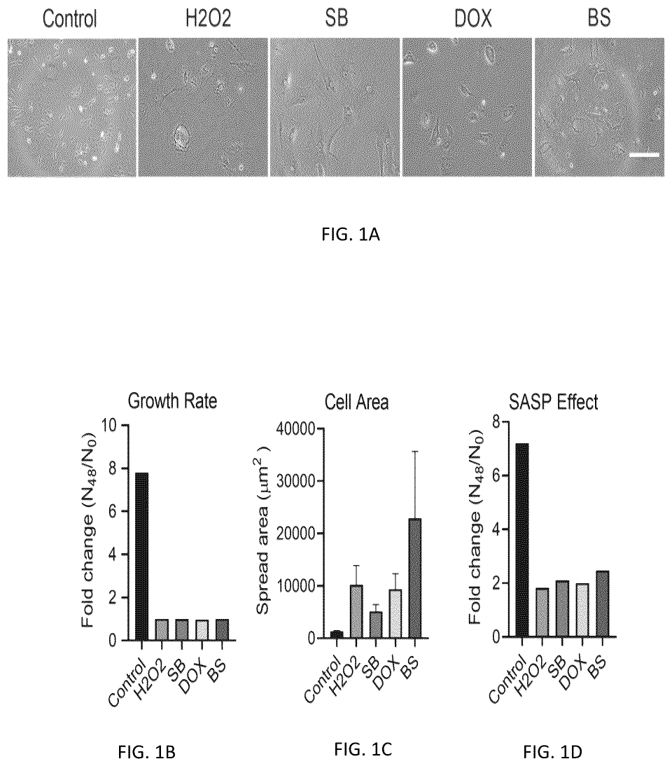

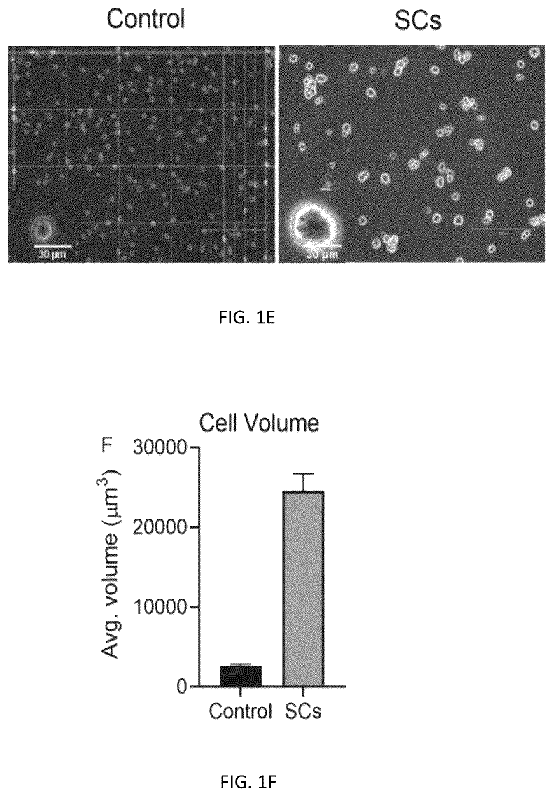

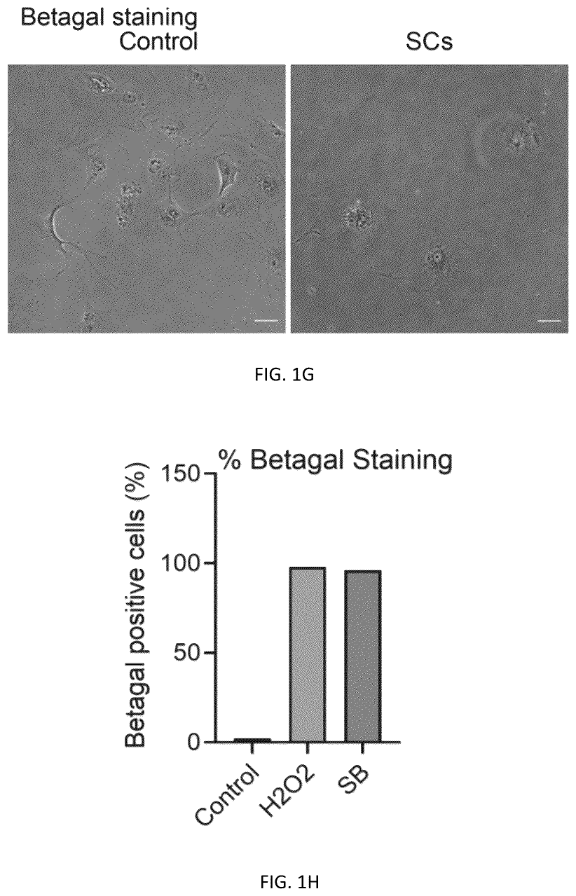

The first studies involved characterizing senescent cells. FIG. 1A shows Brightfield images of control cells and senescent cells. Senescence was induced by H₂O₂, sodium butyrate (SB), Doxorubicin (DOX) or Bleomycin Sulphate (BS) (scale bar=300 μm). FIG. 1B is a bar graph comparing the extent of proliferation. The control cells had approximately an eight-fold change during 48 hours of incubation. In contrast, senescent cells remained non proliferative during the incubation. FIG. 1C is a bar graph comparing the spread area. Senescent cells become enlarged compared to the normal control cells.

Conditioned medium from senescent cells was also used to induce senescence. FIG. 1D is a bar graph comparing proliferation when conditioned medium collected from the various senescent cells: H₂O₂, sodium butyrate (SB), Doxorubicin (DOX), and Bleomycin Sulphate (BS). Medium from each type of treatment inhibited growth of normal proliferating cells compared to the control. FIG. 1. E shows images of control (proliferating) and senescent cells (scale bar=300 μm). FIG. 1F is a bar graph comparing the average volume of control cells with senescent cells (SC). SCs had a significantly larger volume. FIG. 1G shows images with beta galactose staining with is a known marker of cellular senescence. SCs expressed more beta galactose which is evident in the images. FIG. 1H is a bar graph showing the percentage of staining in control cells and senescent cells. Senescence was induced by either H₂O₂ or SB. The SCs show more than 97% beta gal positive cells (scale bar=50 μm).

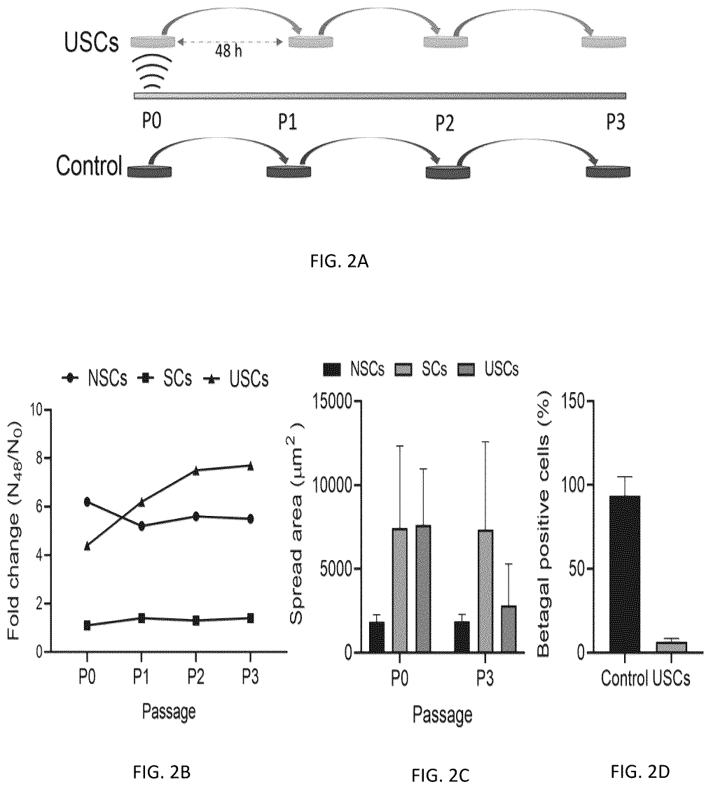

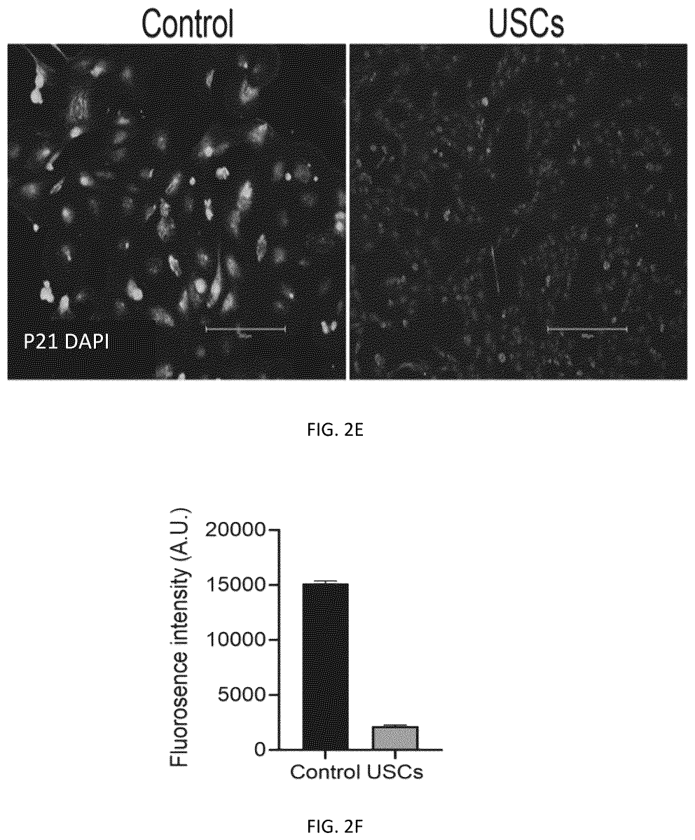

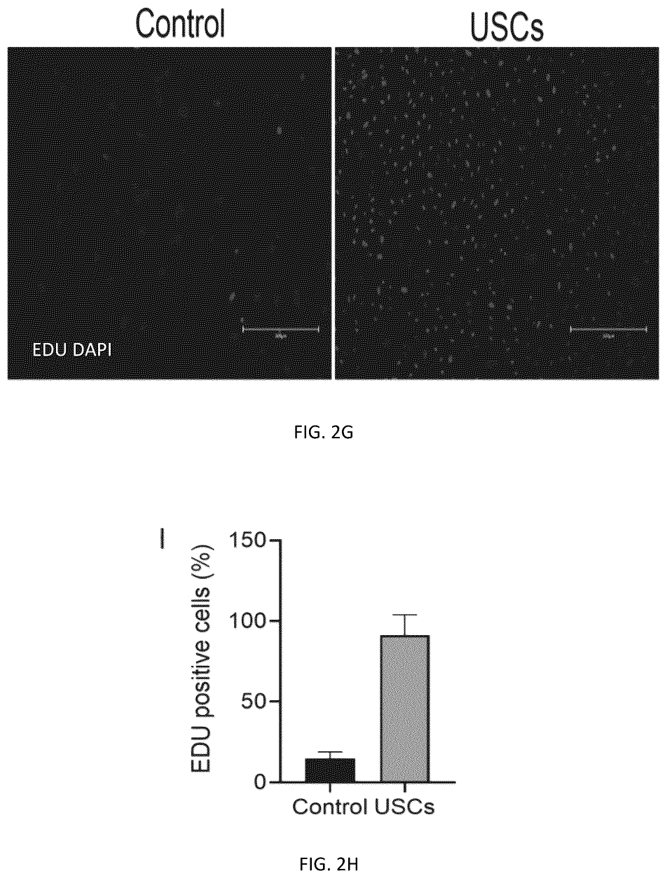

In the next studies, senescent cells were treated with ultrasound. FIG. 2A is a schematic schematic that depicts a SC reversal experiment. SCs were cultured in growth medium for 24 hours. Supernatant was collected before (S0) and 24 hours after ultrasonication treatment (S24). Supernatants S0 and S24 were then used to culture non-senescent control cells.

FIG. 2B is a graph showing the fold change in proliferation of non-senescent cells (NSCs), senescent cells (SCs) and ultrasound treated senescent cells (USCs). The growth rate of ultrasound treated senescent cells (USCs) increased with passages 0-3 while control and senescent cell rates remained relatively constant. Similarly, FIG. 2C is a bar graph comparing the spread area between non-senescent cells (NSCs), senescent cells (SCs) and ultrasound treated senescent cells (USCs). The cell area of USCs was largely restored to the area of normal dividing cells by P3. FIG. 2D is a bar graph showing the percentage of cells that were positive for beta galactose. Ultrasound treated senescent cells (USCs) had almost no detectable beta galactose compared to the control cells.

p21 is a potent cyclin-dependent kinase inhibitor (CKI). FIG. 2E shows images of staining for P21 in control cells and ultrasound treated senescent cells (USCs). FIG. 2F is a bar graph comparing the fluorescence intensity (A.U.) between the control cells and USCs.

Similarly, FIG. 2G shows images of staining for EDU in control cells and USCs. FIG. 2H is a bar graph comparing the percentage of EDU positive cells between the population of control cells and USCs. All graphs were plotted by mean±S.D.

### Example 2

### SASP Inhibits Control, but USS Increases Senescent Cell Growth

In the next studies, the effects of growing non-senescent cells in supernatant taken from senescent cells both pre and post US treatment was investigated. Supernatant was taken from senescent cells before and after US treatment, and this ultrasound supernatant (USS) was introduced to non-senescent cells. Senescent cells normally secrete many pro-inflammatory molecules, growth factors, chemokines, extracellular matrices, proteases and cytokines, collectively known as senescence associated secretary phenotype (SASP).

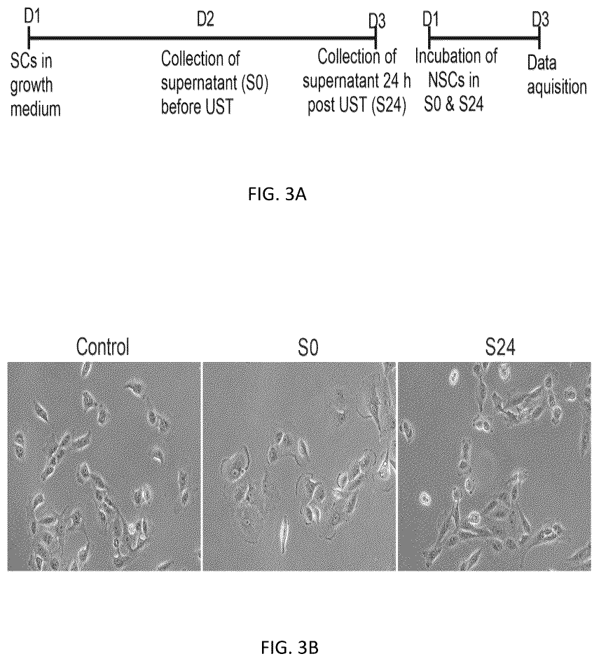

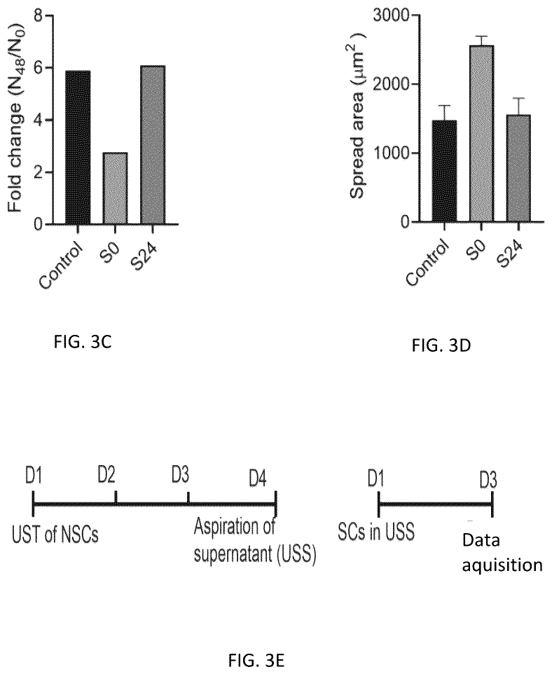

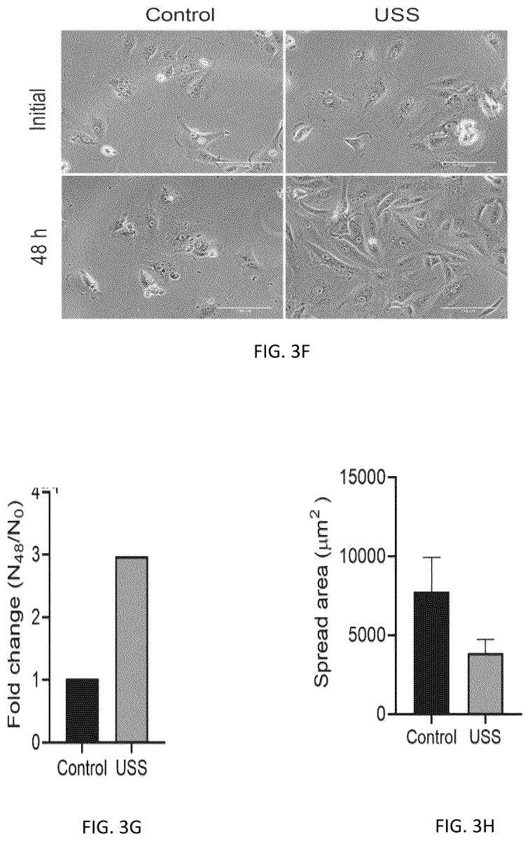

FIG. 3A shows a schematic or timeline of the experiment. SCs were cultured in growth medium for 24 hours. Then, the supernatant from those senescent cells was collected before (S0) and 24 h after ultrasonication treatment (S24). Supernatants S0 and S24 were then used to culture non-senescent control cells. FIG. 3B shows representative images of control cells after 48 hours of incubation in each supernatant.

FIG. 3C and FIG. 3D show the quantification of control cell numbers (C) and cell areas (D) after 48 in control, S0 or S24 supernatants.

For the next studies supernatant was taken from non-senescent cells before and after ultrasound (US) treatment, and this supernatant (USS) was introduced to senescent cells. FIG. 3E is a diagram showing that control, non-senescent, cells were treated with US four times in the same media, and the supernatant was collected (USS) for incubation with senescent cells for 48 hours.

FIG. 3F is a grid of brightfield images comparing the change in morphology of SCs after 48 hours in response to control medium and USS. Senescent cells in normal growth medium were the controls. FIG. 3G shows a quantification of USS increased the growth rate and FIG. 3H shows a quantification of decreased spread area of senescent cells. Graphs were plotted by mean±S.D.

### Example 3

### Ultrasound Decreases Mitochondrial Length and Lysosome Intensity in Senescent Cells

Additional studies characterized the effects of US on mitochondrial length and lysome intensity in senescent cells. Ultrasound treated cells were incubated with Mitotracker (Invitrogen®) in 100 Um at 37° C. for 30 minutes. Then, images were captured in Confocal microscope at 15 randomized fields per sample for quantification. AR and form factors are determined using the formula Major axis/Minor axis and Perimeter 2/(4π×surface area).

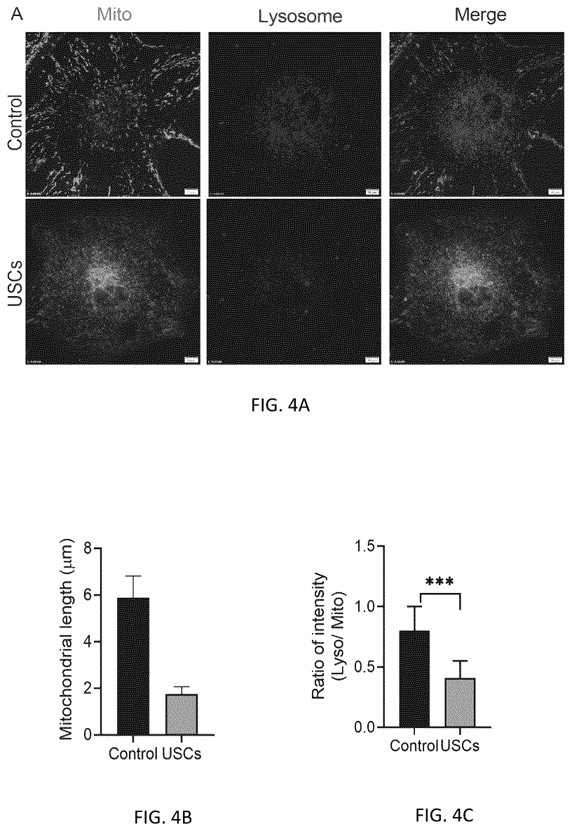

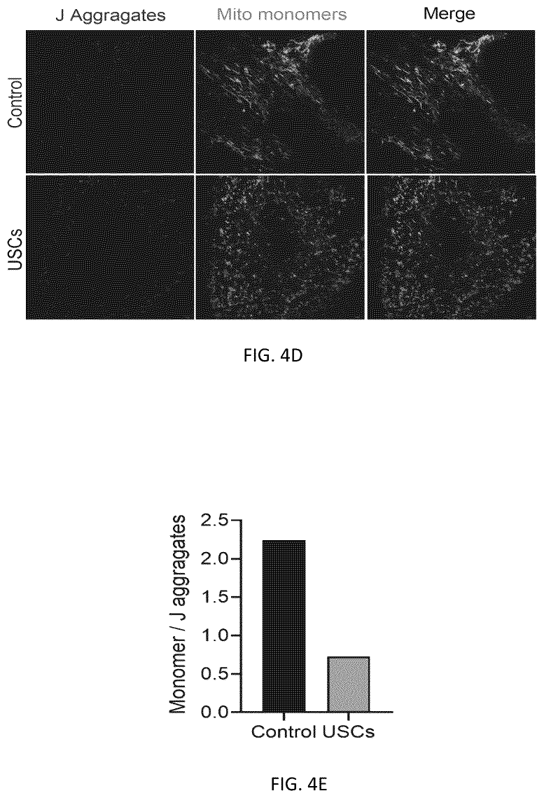

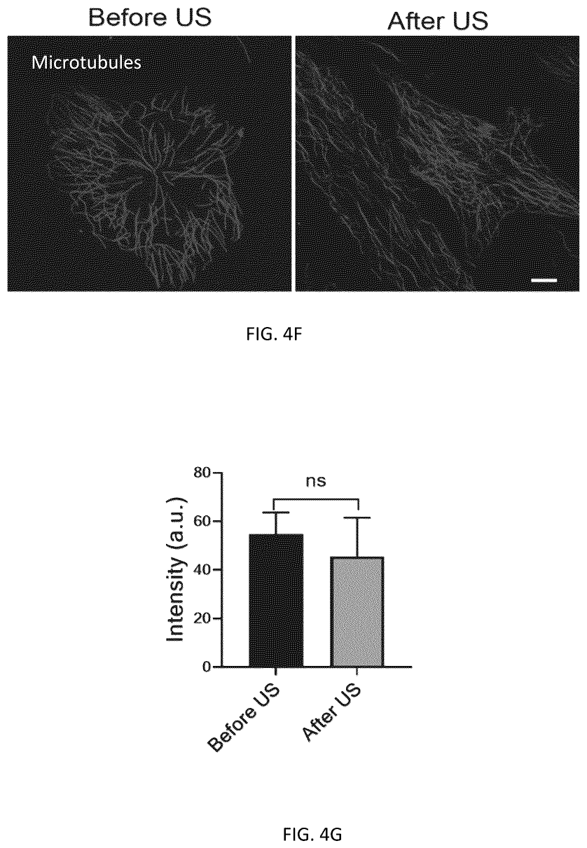

FIG. 4A is a set of immunofluorescent images of mitochondrial morphology and lysosome fluorescence in senescent cells stained with Mitotracker and lysosome tracker (scale bar=10 μm). Ultrasound treated SCs (USCs) have fragmented mitochondria and decreased lysosome staining.

FIG. 4B is a quantification of mitochondrial length showing decreased length after ultrasound irradiation compared to the control cells (n=5, mean±S.D.). FIG. 4C is a quantification of the ratio of intensities of lysosomal to mitochondrial staining, showing that the ratio of intensity is decreased by ultrasound treatment.

FIG. 4D is a set of fluorescence images showing that J aggregates increase in intensity after ultrasound treatment of senescent cells as mitochondria are fragmented (Mitotracker). FIG. 4E is a bar graph comparing the ration of Mito monomers to J aggregates in control cells vs ultra-sound treated cells (USCs). The USCs have a decreased ratio of monomer to J aggregates compared to the control, showing that the J aggregates has increased.

FIG. 4F is a pair of fluorescence images of microtubules in senescent cells before and after ultrasound (scale bar=10 μm). FIG. 4G is a bar graph comparing the Intensity of microtubules in cells before and after ultrasound treatment data for FIG. 4G was quantified from data in 4 F. Data is plotted as mean±S.D., p-value >0.05 is ‘non-significant’ (n=9-10).

### Example 4

### Ultrasound Reversal of Replicative Senescence Increases Number of Cells

In the next set of experiments, the effect of ultrasound treatment and the resulting reversal of replicative senescence and the increase in number of cells was studied.

First senescence was induced in Vero cells using sodium butyrate. Then senescence was confirmed using growth arrest and β-gal staining methods. The senescent cells were treated with ultrasound using ultrasound of optimized parameters (33 kHz frequency and 3.5-4 Pa). These cells were incubated for 48 hours and growth and morphology was measured before the trypsinization. This passage was referred to as “P0.” These cells were then trypsinized, reseeded, and incubated for 48 hours in the P1 passage. This process was repeated to P3 passage. The cells were evaluated in terms of fold change in number for growth, morphology, β-gal, and EDU staining to check the population of senescent cells. Senescent cells without ultrasound treatment were used as control cells. Typically, by passage P3, the senescent cells exhibited the phenotype of normal proliferating cells.

Passage 15-24 HFF cells were treated with ultrasound at the optimized frequency and power. Cell proliferation was determined by counting the number of cells at the time of seeding and 48-hour post US treatment. Ultrasound treated HFF cells showed higher fold change in growth than the untreated HFF cells. In the case of P24 HFF cells, they were treated with ultrasound and incubated for 96 hours prior to trypsinization, reseeding and incubation for 48 hours. Proliferation and morphology were measured after 48 hours of incubation. P24, US treated HFF cells became smaller in size, and they also showed dramatically greater proliferation than the untreated P24 HFF cells.

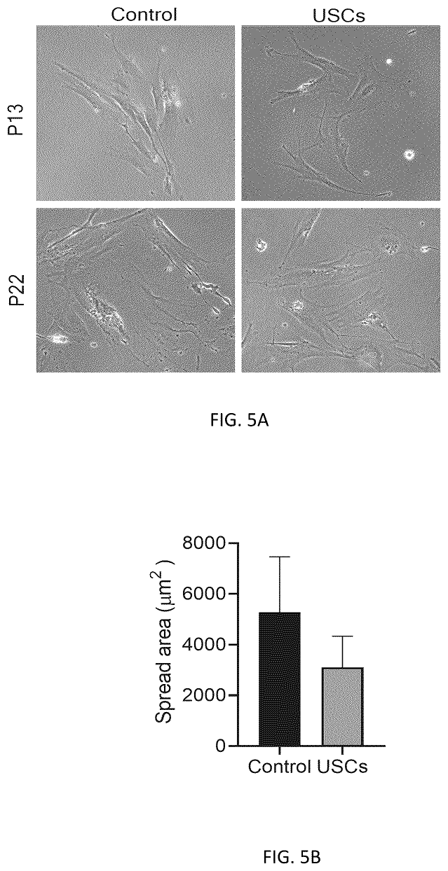

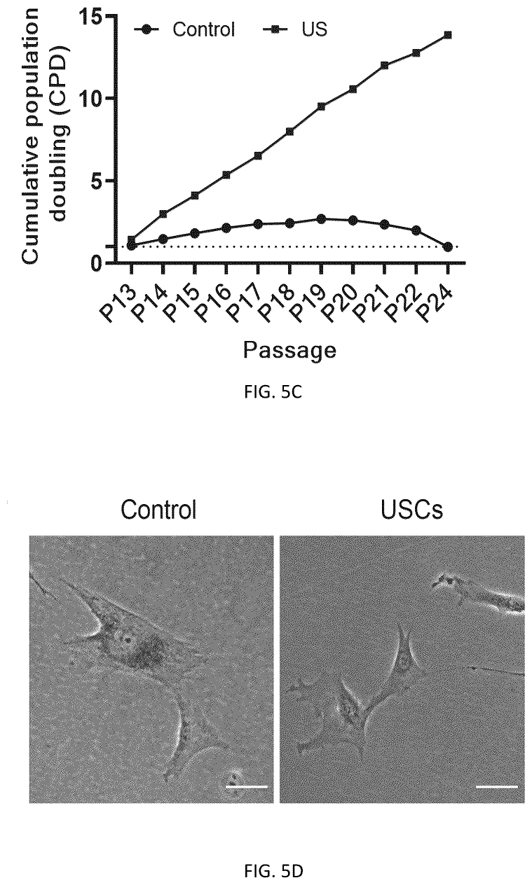

FIG. 5A is a set of Bright field images of untreated control and US treated senescent cells show the morphological changes between P13 and P22 or P24. Control HFF cells were passaged every 48 hours from P13 to P22 passage, while ultrasound rejuvenated cells were US treated at every other passage (scale bar=50 μm). FIG. 5B is a bar graph demonstrating the spread area of P22 or P24 treated control and ultrasound (US) treated cells.

FIG. 5C is a line graph comparing the cumulative population doubling of ultrasound treated senescent cells (USCs) and control cells as passage number increases. Growth rate of ultrasound treated senescent cells (USCs) was higher than the control cells in all late passages. Cumulative population doubling (2×) shows that the population of ultrasound-treated cells is increased by 213-fold relative to the controls.

FIG. 5D is a pair of images comparing the senescence of control and USCs at P22 or P24 checked by Beta gal staining. Senescence of P22 or P24 checked by Beta gal staining. Senescence was indicated by the presence of blue color (i.e., blue dye or blue label).

FIG. 5E is a bar graph comparing the percentage of β-gal positive cells in control and USCs representative images of mitochondria and lysosomes in P6, P13 and P27 cells. The data in FIG. 5E is a quantification of β-gal positive cells from FIG. 5D. The percentage of betagel positive cells decreased in USC treated cells.

### Example 5

### Effects of Ultrasound Treatment on Performance of Aged Mice

Next, the effects of ultrasound treatment on the physical performance of aged mice was investigated. Ultrasound treatment improved the grip strength, inverted cling time, duration of running time, and results for the rotarod test in aged mice.

Aged mice (21-24 months old) were treated in a large (e.g., 4 L) glass beaker with one plastic cylinder. A plastic mess was placed on top the cylinder that supported the mice and enabled them to rest their four limbs and body in the water. Degassed and 32-35° C. warm water was poured into the beaker. The level of water was kept one inch above the plastic mesh so that half of the body of the mice remained in water. Once the mice were placed in the water, intermittent ultrasound of 32.249 kHz and 6-7 kilopascal was applied to the mice for 30′ at 1.5 second on and 1.5 second off. Animals in the ultrasound groups were treated at 72-96 hour intervals for one month (10 treatments). During the ultrasound treatment, mouse activity was carefully observed along with their adaptation to the system. After treatment, the animals were placed in separate cage with tissue paper to dry and then they were returned to their home cage. Control mice were placed in the same water bath for 30′ without ultrasonication. In the ultrasound treatment procedure, animals were in direct contact with the water. The reason for putting animals in water was that ultrasound was attenuated dramatically at air-water interfaces.

There were 12 exercise training sessions and 10 ultrasound treatments in the one-month experimental period.

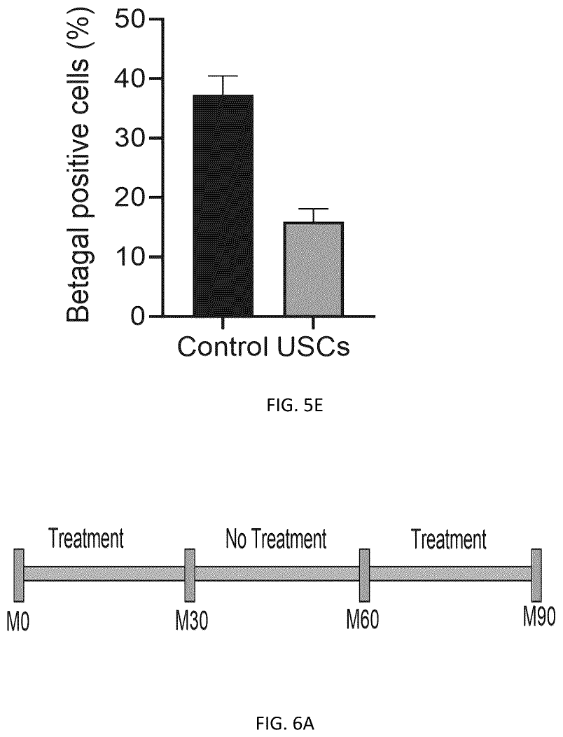

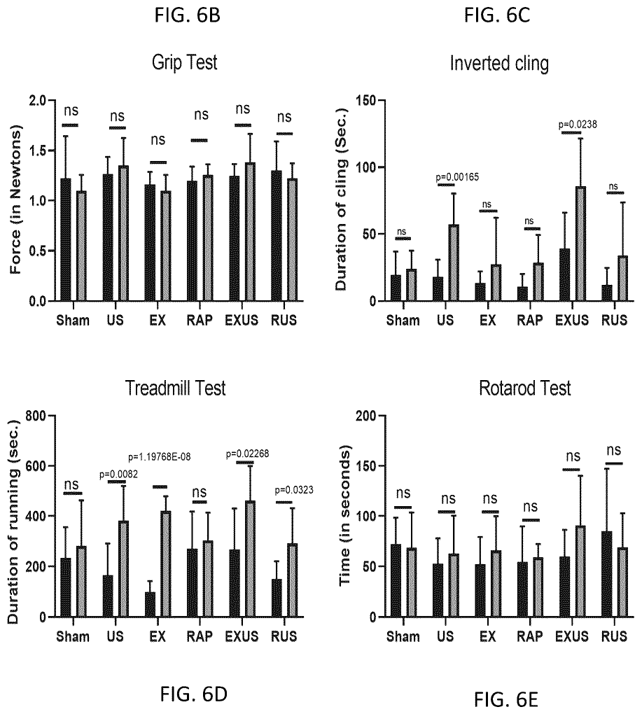

FIG. 6A is a diagram of the treatment protocol for the mice in the ultrasound, exercise, and ultrasound plus exercise groups. 20-24 month old mice (C57BL/6J strain) were treated every 72 hours and exercise training sessions were conducted three times per week. Each study group contained four male and four female mice. FIG. 6B is a bar graph of results of grip tests plotted as the average of the durations. FIG. 6C is a bar graph Graph of results of the Inverted cling test. FIG. 6D is a bar graph of results of the Treadmill tests. FIG. 6E is a bar graph of results of the rotarod tests.

Results are plotted as mean±S.D. A Student t-test was used to determine the statistical significance. P-value greater than 0.05 is represented by ns. Statistical significance was given in p-value.

### Example 6

### Ultrasound has No Effect on Sirtuin1 Inhibitor

Next, the effects of ultrasound on the sirtuin 1 inhibitor was investigated. At a molecular level, LFU rejuvenation of senescent cells was blocked by Sirtuin1 inhibition, EX-527.

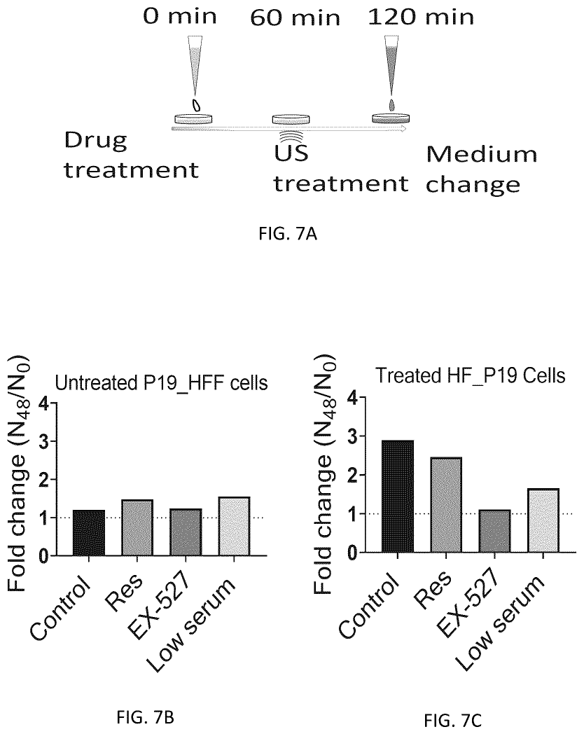

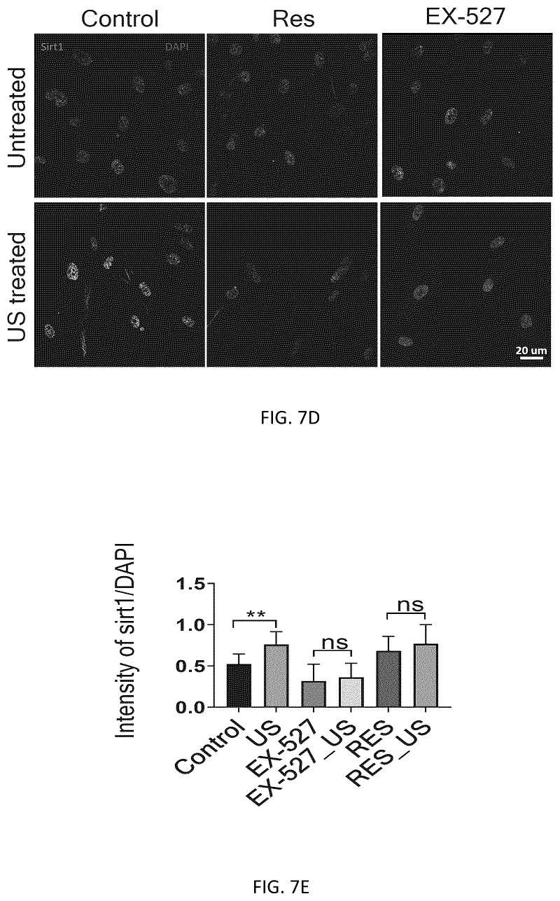

FIG. 7A is a schematic of resveratrol and EX-527 treatment, followed by ultrasound treatment and medium change. FIG. 7B is a bar graph comparing the fold change in cell number of untreated control HFF cells in presence of EX-527.

FIG. 7C is a bar graph comparing the fold change in cell number of treated P19 cells in the presence of drug. FIG. 7D is a set of images comparing the immunofluorescence staining of Sirt1 in untreated and US treated HFF cells. FIG. 7E is bar graph comparing the sirt1 expression by intensity ratio of sirt1 and DAPI. Results are shown as mean±S.D. **p value <0.02 and ‘non-significant’ p value >0.05.

These results support a role for Sirtuin1 in the LFU reversal of senescence since a specific inhibitor of Sirtuin1 blocked the rejuvenation of senescent cells by LFU.

### Example 7

### Low Frequency Ultrasound Increases Activity in Mice

Additional studies were conducted to study the effect of ultrasound on mice. Aged mice were treated with LFU as described above (Ex. 5). The study included three groups of mice (controls, high performers and low performers) that are defined by the level of ultrasound treatment (ranging from 5 hours per month at 1× power to 15 hours per month at 2× power—a 6-fold difference in dosage).

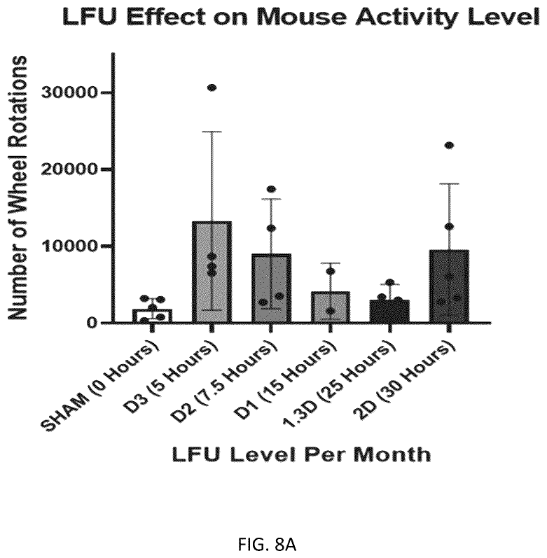

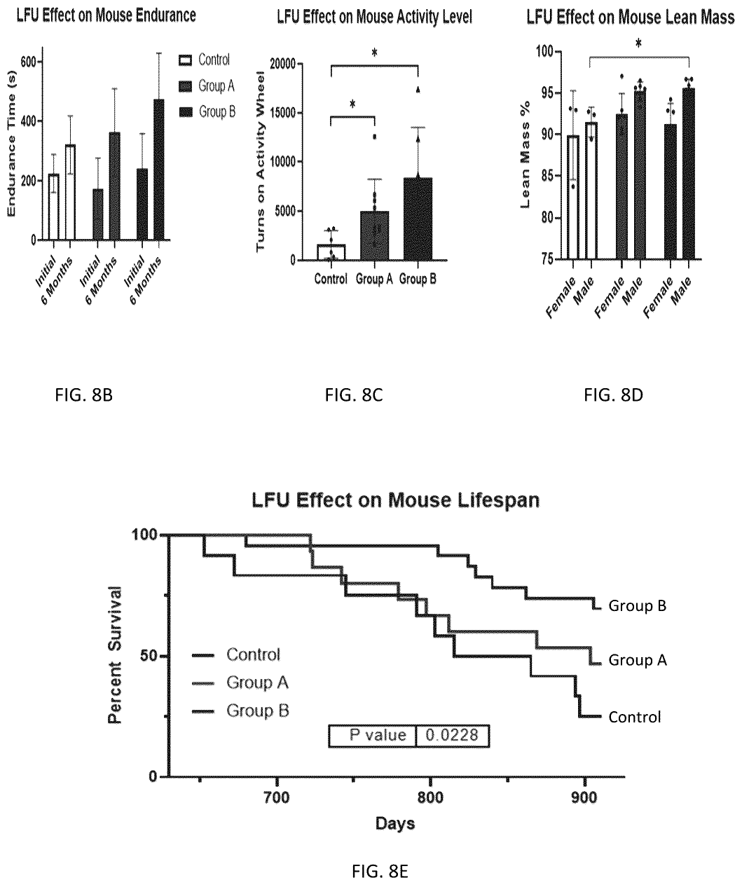

FIG. 8A is a summary of the results of the physical activity (i.e., number of wheel rotations) of five populations of mice:
(a) control or SHAM
(b) D3 with mice treated with LFU for 5 hours with 1× power
(c) D4 with mice treated with LFU for 7.5 hours
(d) D1 with mice treated with LFU for 15 hours
(e) 1.3D with mice treated with LFU for equivalent of 25 hours with 1× power
(f) 2D with mice treated with LFU for equivalent of 30 hours with 1× power

The results demonstrate that ultrasound treated mice were significantly more active than untreated (i.e., control) groups. Moreover, a five-hour treatment was sufficient to increase activity. Additional studies included endurance lean body mass.

FIG. 8B is a bar graph that compares the endurance time of control mice with two groups of mice treated with low frequency ultrasound (LFU). Mice were tested before (initial) and after six months of LFU treatment. Similarly, FIG. 8C is a bar graph that compares the physical activity (i.e., number of wheel rotations) of control mice with two groups of mice (group A and B) treated with low frequency ultrasound (LFU).

FIG. 8D is a bar graph that compares the lean mass of control mice with the same groups of mice. Higher lean mass was observed in both male and female mice treated with LFU.

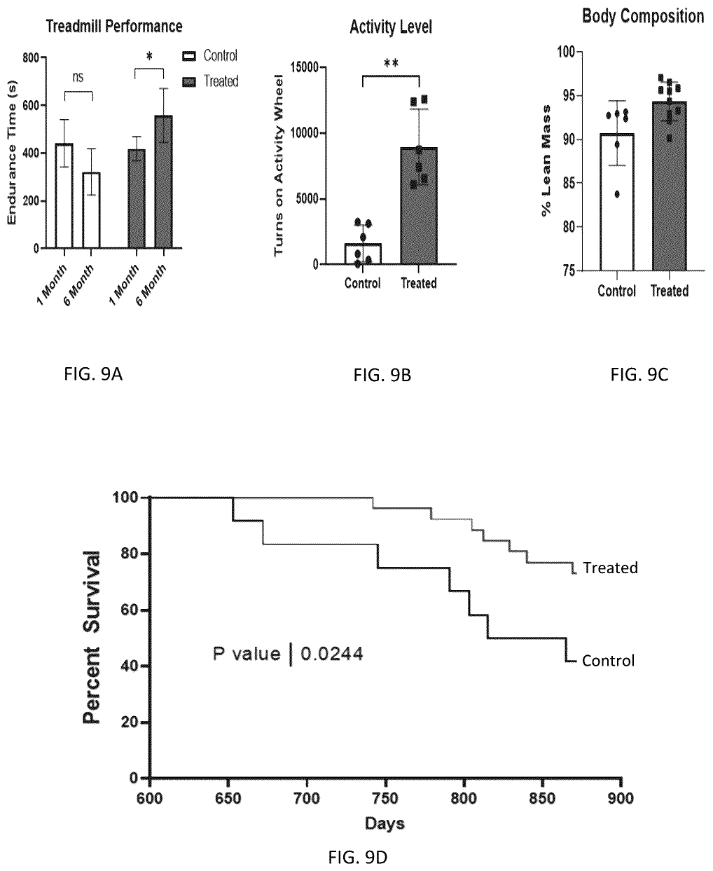

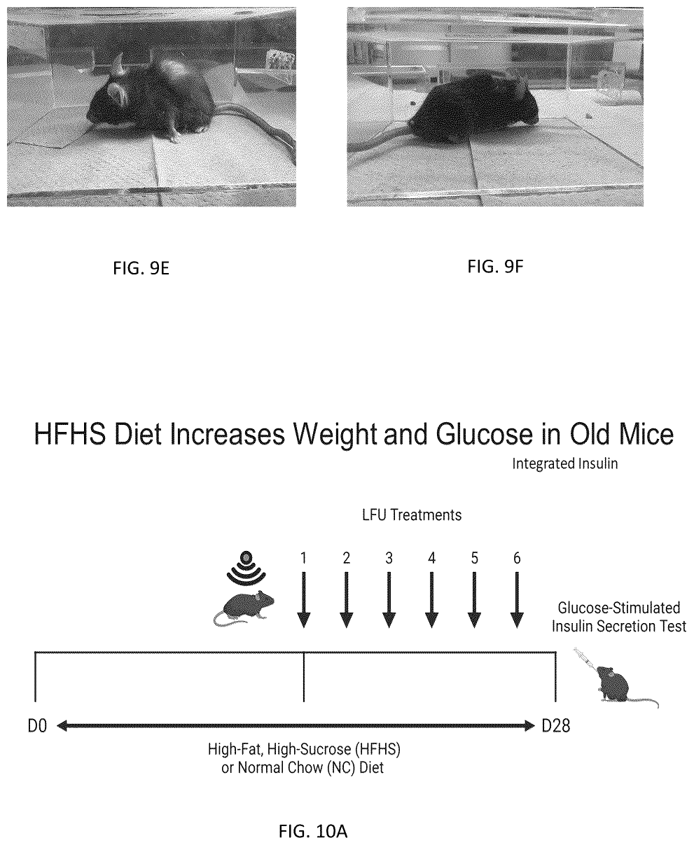

Similarly, FIG. 9A is a bar graph that compares the physical activity (i.e., endurance time on treadmill) of control mice with mice treated with LFU for 1 and 6 months. FIG. 9B is a bar graph that compares the activity level (i.e., turns on an activity wheel) of control mice with the mice treated with LFU. FIG. 9C is a bar graph that compares the body composition (i.e., percent lean mass) of control mice with mice treated with LFU.

### Example 8

### Low Frequency Ultrasound Increases Lifespan of Mice

Additional studies were conducted to study the effect of ultrasound on the life span of mice. FIG. 8E is a graph that compares the survival rate of control mice with two groups of mice (group A and B) treated with ultrasound. The percent survival was significantly higher in the mice treated with LFU. The difference appeared to increase over time. Overall, these experiments demonstrate that LFU increases both activity and lifespan of mice. Similar results are shown in FIG. 9D. It is noted that the increase in lifespan correlated with increased activity.

Mice treated with ultrasound had noticeable differences in appearance. FIG. 9E is a photograph of a control mouse that received thirty-minute baths without ultrasound (LFU) treatment. FIG. 9F is a photograph of a mouse that received two daily treatments of ultrasound (LFU) for six months. Notable differences of treated mice include normal posture, lined and brilliant fur, bright eyes and erect ears in treated mice. As described above, treated mice were also more active. In contrast, the control mice demonstrated significant hair loss and signs of aging/frailty.

### Example 9

### Low Frequency Ultrasound Improves Insulin Response

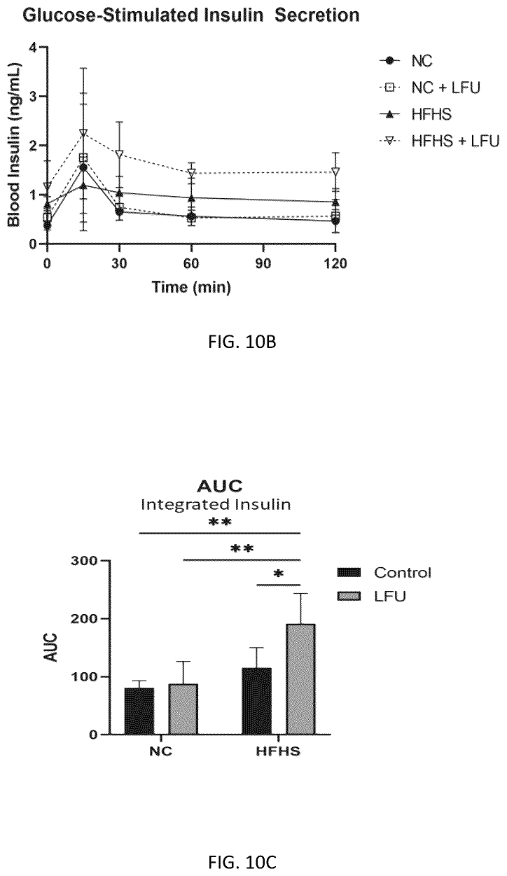

Additional studies were conducted to study the effect of ultrasound on metabolism of mice. FIG. 10A is a schematic representation of an experiment demonstrating that high-fat, high-sucrose (HFHS) diet increases weight and glucose levels in old mice. The effects can be counter-acted by treating the mice with low frequency ultrasound (LFU).

FIG. 10B is a bar graph that compares blood insulin levels of control mice with mice fed a high-fat, high-sucrose (HFHS) diet. Four groups of mice were studied:
a) NC (normal diet, control)
b) NC+LFU (normal diet+ultrasound treatment)
c) HFHS (high-fat, high-sucrose diet)
d) HFHS+LFU (high-fat, high-sucrose diet+ultrasound treatment)
   The HFHS mice treated with low frequency ultrasound (LFU) had near normal levels of blood insulin. In contrast, the control mice had lower insulin levels.

FIG. 10C is a bar graph that compares the integrated insulin (i.e., area under the curve) of control mice (NC) with mice fed a high-fat, high-sucrose (HFHS) diet. Mice from both groups were treated with LFU. The HFHS mice treated with low frequency ultrasound (LFU) had a higher AUC. These experiments demonstrate that LFU increases insulin secretion (likely due to improved pancreas function) in HFHS mice. In the high-fat, high-sucrose data, the level of insulin in the blood was followed for 2 hours after glucose lavage and the integrate area under the curve (AUC) shows that the LFU mice secreted significantly more insulin.

### Example 10

### Low Frequency Ultrasound (LFU) to Treat Osteoarthritis

Osteoarthritis (OA) is a type of degenerative joint disease that results from breakdown of joint cartilage and underlying bone which affects 1 in 7 adults in the United States. The most common symptoms are joint pain and stiffness. Damage from mechanical stress with insufficient self-repair by joints is believed to be the primary cause of osteoarthritis. The risk of osteoarthritis increases with ageing. Conventional treatments include exercise, decreasing joint stress such as by rest or use of a cane, support groups and pain medications.

In this example, patient (65-year-old female) visits a physicians' clinic and presents signs and symptoms of osteoarthritis. Specifically, the patient presents signs/symptoms of chronic inflammation/osteoarthritis in the joints of her hands and fingers. A healthcare provider suspects that the targeted rejuvenation of senescent cells will improve the patient's overall health.

The patient is treated with LFU that target cells in her hands/fingers. Specifically, intermittent ultrasound of 32.249 kHz and 6-7 kilopascal is applied to the hands for 30 minutes at 1.5 second on and 1.5 second off. The patient receives daily treatments for five days (five treatments in total).

The LFU rejuvenates senescent cells in the hands/fingers of the patient. One week after treatment, the signs/symptoms of osteoarthritis are reduced by approximately 70%. The health care provider also notes that inflammation appears absent. The healthcare provider recommends weekly LFU treatments and continues to monitor the patient's inflammation and osteoarthritis. After the treatment, the patient expresses relief of pain from inflammation.

### Example 11

### Low Frequency Ultrasound (LFU) to Treat Diabetes

Cells within the pancreas help to maintain blood glucose levels (i.e., homeostasis). The cells that do this are located within the pancreatic islets that are present throughout the pancreas. When blood glucose levels are low, alpha cells secrete glucagon, which increases blood glucose levels. When blood glucose levels are high beta cells secrete insulin to decrease glucose in blood. Delta cells in the islet also secrete somatostatin which decreases the release of insulin and glucagon.

Diabetes mellitus type 2 (T2) is the most common form of diabetes. The causes for high blood sugar in this form of diabetes usually are a combination of insulin resistance and impaired insulin secretion, with both genetic and environmental factors playing a role in the development of the disease. Over time, pancreatic beta cells may become “exhausted” and less functional.

In this example, patient (65-year-old male) visits a physicians' clinic and presents signs and symptoms of T2 diabetes. Specifically, the patient presents elevated levels of blood glucose and low levels of circulating insulin. A healthcare provider suspects that the targeted rejuvenation of senescent cells in the pancreas will improve the patient's metabolism and overall health.

The patient is treated with LFU that targets cells in the pancreas. Specifically, intermittent ultrasound of 32.249 kHz and 6-7 kilopascal is applied to the abdomen for 30 minutes at 1.5 second on and 1.5 second off. The patient receives daily treatments for five days (five treatments in total).

The LFU rejuvenates senescent cells in the pancreas. One week after treatment, the signs/symptoms of T2 diabetes are reduced by approximately 75%. Levels of blood glucose are normal along with levels of circulating insulin. The healthcare provider recommends weekly LFU treatments and continues to monitor the patient.

### Example 12

### Low Frequency Ultrasound (LFU) to Hair Loss

Hair loss, also known as alopecia or baldness, refers to a loss of hair from part of the head or body. Typically, at least the head is involved. Common types include male- or female-pattern hair loss, alopecia areata, and a thinning of hair known as telogen effluvium. The cause of male-pattern hair loss is a combination of genetics and male hormones. In this example, patient (45-year-old male) visits a physicians' clinic with noticeable hair loss to his scalp.

The patient is treated with LFU that targets dermal cells. Specifically, intermittent ultrasound is applied to the top of the head for 30 minutes at 1.5 second on and 1.5 second off. A low level i.e., (5-20 kHz) is used to ensure that the LFU does not penetrate the skin. The patient receives daily treatments for 30 days.

The LFU rejuvenates dermal cells and improves hair growth. Two to three weeks after treatment, significant hair growth is observed. Within two months, most (i.e., about 70%) of his hair has returned. The healthcare provider recommends weekly LFU treatments and continues to monitor the patient.

### Example 13

### Low Frequency Ultrasound (LFU) to Promote Wound Healing

In aspects, pulsed ultrasound can be used to (a) improve wound healing, (b) improve bone/tissue healing, (c) improve recovery from injury and (d) improve perfusion to tissue. In this example, a patient suffers from a torn meniscus. The patient undergoes arthroscopic surgery to repair the cartilage. Following the procedure (i.e., more than 24 hours later), the patient is treated with pulsed ultrasound to promote healing and reduce recovery time.

Specifically, the patient is treated with LFU that targets cartilage cells and other tissue that was affected by the surgery. Specifically, intermittent ultrasound of 32.249 kHz and 6-7 kilopascal is applied to the knee for 30 minutes at 1.5 second on and 1.5 second off. The patient receives daily treatments for five days (five treatments in total).

One week after treatment, the knee is almost completely healed. The patient reports minimal pain and almost no noticeable scarring from the incision. The healthcare provider recommends LFU treatments twice per week until the knee is completely healed.

### Methods of Treatment—Generally

Methods of the present invention include modulating cellular activity by providing ultrasound waves to cells or tissues at an effective intensity and for effective time range so to induce mechanical stresses. The methods described herein utilize low levels of ultrasound. Ultrasound has been approved for human treatment exposure at power density levels (flux levels) ten to hundred-fold higher than the levels used in the methods described herein.

Accordingly, in embodiments, any frequency of ultrasonic radiation between 0.03 MHz to 10 MHz is utilized. In a further embodiment, low frequency ultrasound (LFU), low intensity low frequency ultrasound (LILFU) or low intensity pulsed ultrasound (LIPU) is administered.

Accordingly, the present invention includes methods of using low-intensity (<500 mW/cm²), low-frequency ultrasound (<0.9 MHz). For example, low intensity can be about 450 mW/cm², about 400 mW/cm², about 350 mW/cm², about 300 mW/cm², about 250 mW/cm², about 200 mW/cm², about 150 mW/cm², about 100 mW/cm², about 50 mW/cm², about 25 mW/cm², about 10 mW/cm², and levels of ultrasound intensity within these stated amounts, including from about 450 mW/cm² to about 1 mW/cm².

Low frequency ultrasound may include ranges from about 0.88 MHz to about 0.01 MHz, from about 0.80 MHz to about 0.01 MHz, 0.80 MHz to about 0.1 MHz, from about 0.70 MHz to about 0.1 MHz, from about 0.60 MHz to about 0.1 MHz, from about 0.50 MHz to about 0.1 MHz, from about 0.40 MHz to about 01 MHz, from about 0.30 MHz to about 0.1 MHz, from about 0.20 MHz to about 0.1 MHz, from about 0.10 MHz to about 0.1 MHz, and levels of ultrasound frequency within these stated amounts.

In an embodiment, the ultrasound is applied with pressure pulses of approximately 170 pa, 340 pa, 510 pa, 680 pa, 850 pa, 990 pa, 1,130 pa, 1,300 pa, 1,470 pa, 1,640 pa, 1,810 pa, 1,980 pa, 2,150 pa, 2,320 pa, 2,490 pa, 2,660 pa, 2,830 pa, 3,000 pa, 3,500 pa, 4,000 pa, 4,500 pa or more.

In an embodiment, the interval of administration of a therapeutic method is anywhere from once to any number of times a second, minute, hour, day, week, month, or year, or more, or continuously for any number of seconds, minutes, hours, days, weeks, months, years, or more.

In an embodiment, ultrasound is administered for a period of approximately: less than 1 second, 1 second, 5 seconds, 10 seconds, 15 seconds, 20 seconds, 25 seconds, 30 seconds, 35 seconds, 40 seconds, 45 seconds, 50 seconds, 55 seconds, 1 minute, 2, minutes, 3 minutes, 4 minutes, 5 minutes, 6 minutes, 7 minutes, 8 minutes, 9 minutes, 10 minutes, 15 minutes, 20 minutes, 25 minutes, 30 minutes, 40 minutes, 45 minutes, 50 minutes 55 minutes, 1 hour, 1.5 hours, 2 hours, 2.5 hours, 3 hours, 3.5 hours, 4 hours, 4.5 hours, 5 hours, 6 hours, 7 hours, 8, hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more.

In embodiments, ultrasound is administered to a subject in cycles. Ultrasound is administered for a specific period of time (e.g., 1.5 seconds) followed by a rest period (e.g., 1.5 seconds). This cycle can be repeated for the duration of a treatment (e.g., two minutes to two hours). The cycle can be repeated daily, for example, over the course of two days to thirty days.

In a further embodiment, a period of during which administration is stopped is for approximately: less than 1 second, 1 second, 5 seconds, 10 seconds, 15 seconds, 20 seconds, 25 seconds, 30 seconds, 35 seconds, 40 seconds, 45 seconds, 50 seconds, 55 seconds, 1 minute, 2, minutes, 3 minutes, 4 minutes, 5 minutes, 6 minutes, 7 minutes, 8 minutes, 9 minutes, 10 minutes, 15 minutes, 20 minutes, 25 minutes, 30 minutes, 40 minutes, 45 minutes, 50 minutes 55 minutes, 1 hour, 1.5 hours, 2 hours, 2.5 hours, 3 hours, 3.5 hours, 4 hours, 4.5 hours, 5 hours, 6 hours, 7 hours, 8, hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In a further embodiment, the ultrasonic radiation is administered whereby the radiation is switched on and off periodically throughout the treatment process.

In embodiments, ultrasound is administered to a subject in modulated cycles. Ultrasound is administered at a specific intensity for a first period (e.g., 1.5 seconds) followed by reduced intensity for a second period (e.g., 1.5 seconds). This cycle can be repeated for the duration of a treatment (e.g., two minutes to two hours). The cycle can be repeated daily, for example, over the course of two days to thirty days. In aspects, the first period is 2 seconds and the second period is 2 seconds. In aspects, the first period is 5 seconds and the second period is 5 seconds. In aspects, the first period is 10 seconds and the second period is 10 seconds. In aspects, the first period is 20 seconds and the second period is 20 seconds. In aspects, the first period is 30 seconds and the second period is 30 seconds. In aspects, the first period is 60 seconds or more and the second period is 60 seconds or more. In aspects, the first period and the second period are different (e.g., a longer low intensity cycle).

In embodiments, the modulation can be adjusted over the course of a treatment. For example, the rest period can increase or decrease over the course of treatment. The power level can also be adjusted over time. In aspects, methods include longer rest periods to promote recovery or healing.

In a further embodiment, the total duration in which the stressor (e.g., ultrasonic radiation) is applied to a subject (e.g., cells, animal, or human patient) is less than 1 day, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, 1 year, 1.5 years, 2 years, 2.5 years, 3 years, 3.5 years, 4 years, 4.5 years, 5 years or more.

### Methods of Treatment—Cell Cultures

In an embodiment, the distance between the cell culture and an ultrasound transducer is directly in contact, or approximately separated from the cell culture sample by 1 cm, 2 cm, 3 cm, 4 cm, 5 cm, 6 cm, 7 cm, 8 cm, 9 cm, 10 cm, 12 cm, 13 cm, 14 cm, 15 cm, 16 cm, 17 cm, 18 cm, 19 cm, 20 cm, 25 cm, 30 cm, 35 cm, 40 cm, 45 cm, 50 cm, 55 cm, 60 cm, 65 cm, 70, 75, 80, 85, 90, 95, 100 cm, or more.

In one embodiment, a therapeutic method disclosed herein can reduce, stop, or reverse cellular senescence in in vitro cell cultures by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95% as compared to cells not receiving the same treatment. In other aspects of this embodiment, a therapeutic method is capable of reducing the number of signs/symptoms of a senescence in cells by, e.g., about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70% as compared to cells not receiving the same treatment.

In an embodiment, senescent cells are treated with a stressor (e.g., ultrasonic radiation) in order to slow, stop, or reverse cell cycle arrest by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95% as compared to sodium butyrate induced senescent cells not receiving the same treatment. In other aspects of this embodiment, senescent cells can be treated with a stressor (e.g., ultrasonic radiation) to reduce, stop, or reverse cellular senescence by, e.g., about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%, as compared to senescent cells not receiving the same treatment.

In an embodiment, sodium butyrate induced senescent cells can be treated with a stressor (e.g., ultrasonic radiation) to reduce, stop, or reverse cellular senescence by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95% as compared to sodium butyrate induced senescent cells not receiving the same treatment. In other aspects of this embodiment, sodium butyrate induced senescent cells can be treated with ultrasound therapy to reduce, stop, or reverse cellular senescence by, e.g., about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%, as compared to sodium butyrate induced senescent cells not receiving the same treatment.

In an embodiment, senescent cells can be treated with a stressor (e.g., ultrasonic radiation) to reduce, stop, or reverse cellular senescence by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95% as compared to senescent cells not receiving the same treatment. In other aspects of this embodiment, senescent cells can be treated with a stressor (e.g., ultrasonic radiation) to reduce, stop, or reverse cellular senescence by, e.g., about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%, as compared to senescent cells not receiving the same treatment.

In an embodiment, serial passage induced senescent cells can be treated with a stressor (e.g., ultrasonic radiation) to reduce, stop, or reverse cellular senescence by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95% as compared to sodium butyrate induced senescent cells not receiving the same treatment. In other aspects of this embodiment, serial passage induced senescent cells can be treated with ultrasound therapy to reduce, stop, or reverse cellular senescence by, e.g., about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%, as compared to, serial passage induced senescent cells not receiving the same treatment.

In an embodiment, senescent cells are treated with a stressor (e.g., ultrasonic radiation) in order to reduce, stop, or reverse the enlargement of mitochondria, a phenotype of cellular senescence, by at least 1%, 2%, 3%, 4%, 5%, 6%, 7%, 8%, 9%, 10%, 11%, 12%, 13%, 14%, 15%, 16%, 17%, 18%, 19%, 20%, 25%, 26%, 30%, 35%, 40%, 45%, 50%, 55%, 60%, 65%, 70%, 75%, 80%, 90%, 95%, 100%. In other aspects of this embodiment, senescent cells are treated with a stressor (e.g., ultrasonic radiation) in order to reduce, stop, or reverse the enlargement of mitochondria, a phenotype of cellular senescence, by, e.g., about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%, as compared to the enlarged mitochondria of senescent cells not receiving the same treatment.

In an embodiment, senescent cells are treated with a stressor (e.g., ultrasonic radiation) in order to reduce β-gal activity (increased β-gal activity is a senescence associated secretory phenotype), a phenotype of cellular senescence, by at least 1%, 2%, 3%, 4%, 5%, 6%, 7%, 8%, 9%, 10%, 11%, 12%, 13%, 14%, 15%, 16%, 17%, 18%, 19%, 20%, 25%, 26%, 30%, 35% 40% 45% 50% 55%, 60%, 65%, 70%, 75% 80%, 90%, 95%, 100%. In other aspects of this embodiment, senescent cells are treated with ultrasound in order to reduce β-gal activity (increased β-gal activity is a senescence associated secretory phenotype), a phenotype of cellular senescence, by, e.g., about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%, as compared to the in order to reduce β-gal activity of senescent cells not receiving the same treatment.

In an embodiment, senescent cells are treated with a stressor (e.g., ultrasonic radiation) in order to slow, stop, or reduce SASP (senescence associated secretory phenotype), by at least 1%, 2%, 3%, 4%, 5%, 6%, 7%, 8%, 9%, 10%, 11%, 12%, 13%, 14%, 15%, 16%, 17%, 18%, 19%, 20%, 25%, 26%, 30%, 35% 40% 45% 50% 55%, 60%, 65%, 70%, 75% 80%, 90%, 95%, 100%. In other aspects of this embodiment, senescent cells are treated with ultrasound in order to slow, stop, or reduce SASP, a phenotype of cellular senescence, by, e.g., about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%, as compared to the SASP of senescent cells not receiving the same treatment.

In an embodiment, senescent cells are treated with a stressor (e.g., ultrasonic radiation) in order to slow, stop, or reduce increased cell size, a phenotype of cellular senescence, by at least 1%, 2%, 3%, 4%, 5%, 6%, 7%, 8%, 9%, 10%, 11%, 12%, 13%, 14%, 15%, 16%, 17%, 18%, 19%, 20%, 25%, 26%, 30%, 35% 40% 45% 50% 55%, 60%, 65%, 70%, 75% 80%, 90%, 95%, 100%. In other aspects of this embodiment, senescent cells are treated with a stressor (e.g., ultrasonic radiation) in order to slow, stop, or reduce increased cell size, a phenotype of cellular senescence, by, e.g., about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%, as compared to the senescent cells not receiving the same treatment.

In an embodiment, cells are treated with a stressor (e.g., ultrasonic radiation) and the supernatant from the treated cells is introduced to other cells to slow, stop, or reverse senescence in the other cell culture by at least 1%, 2%, 3%, 4%, 5%, 6%, 7%, 8%, 9%, 10%, 11%, 12%, 13%, 14%, 15%, 16%, 17%, 18%, 19%, 20%, 25%, 26%, 30%, 35% 40% 45% 50% 55% 60% 65% 70% 75% 80%, 90%, 95%, 100% or more. In this embodiment, the percentage of supernatant from the ultrasound treated cells compared to the fresh medium to be introduced to the untreated cells is at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35% 40% 45% 50% 55% 60% 65% 70%, 75% 80%, 90%, 95%, 100%.

In an embodiment, cells are treated with a stressor (e.g., ultrasonic radiation) along with a therapeutically effective amount of Rho Kinase inhibitor, which slows, reverses, or stops cellular senescence by at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35% 40% 45% 50% 55% 60% 65% 70% 75% 80%, 90%, 95%, 100%.

### Methods of Use on Organisms and Tissues—Disorders, Transplantation, and Physical Performance in Organisms

In an embodiment, the a stressor (e.g., ultrasonic radiation) is applied: directly to the patient, using a transducer or applicator or probe, or other suitable ultrasonic delivery method, that is in direct contact with the patient's skin or tissue, to tissues or fluids removed from the patient and transplanted back into the patient or another patient, to a medium that the patient or the part of the patient is least 1%, 2%, 3%, 4%, 5%, 6%, 7%, 8%, 9%, 10%, 11%, 12%, 13%, 14%, 15%, 16%, 17%, 18%, 19%, 20%, 25%, 26%, 30%, 35%, 40%, 45% 50%, 55%, 60%, 65% 70%, 75%, 80%, 90%, 95%, 100% immersed in or covered by, or held by 1 cm, 2 cm, 3 cm, 4 cm, 5 cm, 6 cm, 7 cm, 8 cm, 9 cm, 10 cm, 12 cm, 13 cm, 14 cm, 15 cm, 16 cm, 17 cm, 18 cm, 19 cm, 20 cm, 25 cm, 30 cm, 35 cm, 40 cm, 45 cm, 50 cm, 55 cm, 60 cm, 65 cm, 70, 75, 80, 85, 90, 95, 100 cm, or more away from the patient. In this embodiment, the “medium” may be any therapeutically suitable solid, liquid, gas, colloid, gel, or other state of matter.

In an embodiment, a stressor (e.g., ultrasonic radiation) is applied to donor organs or fluids prior to, during, and after transplantation into a host patient, where the treatment increases the successful acceptance of the donor organ or fluids and promote healing by at least 1%, 2%, 3%, 4%, 5%, 6%, 7%, 8%, 9%, 10%, 11%, 12%, 13%, 14%, 15%, 16%, 17%, 18%, 19%, 20%, 25%, 26%, 30%, 35% 40% 45% 50% 55%, 60%, 65%, 70%, 75% 80%, 90%, 95%, 100%. In other aspects of this embodiment, donor organs or fluids are treated with ultrasound prior to, during, and after transplantation to increase the chance of successful acceptance of the donor organs and fluids and promote healing, by, e.g., about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%, as compared to the size of senescent cells not receiving the same treatment.

In one embodiment, a therapeutic amount a stressor (e.g., ultrasonic radiation) is applied to donor blood or other donor fluids, then a therapeutic amount of these donor fluids are then administered to a recipient wherein the treatment disclosed herein can reduce the signs/symptoms of a senescence-associated disease or disorder by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95% as compared to a patient not receiving the same treatment. In other aspects of this embodiment, the therapeutic method is capable of reducing the number of signs/symptoms of a senescence-associated disease or disorder in an individual by, e.g., about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70% as compared to a patient not receiving the same treatment.

In one embodiment, a therapeutic method disclosed herein can reduce the signs/symptoms of a senescence-associated disease or disorder by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95% as compared to a patient not receiving the same treatment. In other aspects of this embodiment, a therapeutic method is capable of reducing the number of signs/symptoms of a senescence-associated disease or disorder in an individual by, e.g., about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70% as compared to a patient not receiving the same treatment.

In one embodiment, a therapeutic method disclosed herein is capable of reducing signs/symptoms in an individual or organism suffering from a senescence-associated disease or disorder by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95% as compared to a patient not receiving the same treatment. In other aspects of this embodiment, a method is capable of reducing signs/symptoms in an individual suffering from a senescence-associated disease or disorder by, e.g., about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70% as compared to a patient not receiving the same treatment.

In one embodiment, a therapeutic method disclosed herein is capable of improving the physical performance of an organism or patient by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95% as compared to an organism or patient not receiving the same treatment. In other aspects of this embodiment, a therapeutic method is capable of improving the physical performance by, e.g., about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70% as compared to a patient not receiving the same treatment.

In one embodiment, a stressor (e.g., ultrasonic radiation) is applied to a patient along with a regular exercise regimen. In a further embodiment, the length of time for each session of physical exercise lasts for at least 1 min, 5 min, 10 min, 15 min, 20 min, 25 min, 30 min, 35 min, 40 min, 45 min, 50 min, 55 min, 60 min, 75 min, 90 min, 100 min, 120 min, 150 min, 180 min or more.

In a further embodiment, the frequency in which the patient exercises would be once every 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 35 days, 40 days, 45 days, 50 days, 55 days, 60 days, 65 days, 70 days, 75 days, 80 days, 85 days, 90 days, or more.

In a further embodiment, the total duration in which the patient adheres to the exercise regimen would be 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, 1 year, 1.5 years, 2 years, 2.5 years, 3 years, 3.5 years, 4 years, 4.5 years, 5 years or more.

In one embodiment, a therapeutic method disclosed herein is capable of reducing, stopping, or reversing signs/symptoms of neurodegeneration or a neurodegenerative disease by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95% as compared to a patient not receiving the same treatment. In other aspects of this embodiment, a therapeutic is capable of reducing signs/symptoms in an individual suffering from a senescence-associated disease or disorder by e.g., about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70% as compared to a patient not receiving the same treatment.

In one embodiment, a therapeutic method disclosed herein is capable of reducing, stopping, or reversing signs/symptoms in an individual or organism suffering from an ailment (e.g., a neurodegeneration or a neurodegenerative) disease by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95% as compared to a patient not receiving the same treatment. In other aspects of this embodiment, a therapeutic is capable of reducing signs/symptoms in an individual suffering from a senescence-associated disease or disorder by, e.g., about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70% as compared to a patient not receiving the same treatment.

In aspects of this embodiment, a therapeutically effective amount of a stressor (e.g., ultrasonic radiation) described herein reduces signs/symptoms in an individual suffering from a senescence-associated disease or disorder by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 100%. In other aspects of this embodiment, a therapeutically effective amount of low frequency ultrasound (LFA) disclosed herein reduces signs/symptoms by, e.g., at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, at most 95% or at most 100%. In yet other aspects of this embodiment, a therapeutically effective amount of a stressor (e.g., ultrasonic radiation) reduces signs/symptoms by, e.g., about 10% to about 100%, about 10% to about 90%, about 10% to about 80%, about 10% to about 70%, about 10% to about 60%, about 10% to about 50%, about 10% to about 40%, about 20% to about 100%, about 20% to about 90%, about 20% to about 80%, about 20% to about 20%, about 20% to about 60%, about 20% to about 50%, about 20% to about 40%, about 30% to about 100%, about 30% to about 90%, about 30% to about 80%, about 30% to about 70%, about 30% to about 60%, or about 30% to about 50%.

In other aspects, a therapeutically effective amount of a stressor (e.g., ultrasonic radiation) disclosed herein reduces the aging process and/or reduces signs of aging in an individual by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 100%. In other aspects of this embodiment, a therapeutically effective amount of a stressor (e.g., ultrasonic radiation) disclosed herein reduces the aging process and/or reduces signs of aging by, e.g., at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, at most 95% or at most 100%. In yet other aspects of this embodiment, a therapeutically effective amount of low frequency ultrasound (LFA) disclosed herein reduces the aging process and/or reduces signs of aging by, e.g., about 10% to about 100%, about 10% to about 90%, about 10% to about 80%, about 10% to about 70%, about 10% to about 60%, about 10% to about 50%, about 10% to about 40%, about 20% to about 100%, about 20% to about 90%, about 20% to about 80%, about 20% to about 20%, about 20% to about 60%, about 20% to about 50%, about 20% to about 40%, about 30% to about 100%, about 30% to about 90%, about 30% to about 80%, about 30% to about 70%, about 30% to about 60%, or about 30% to about 50%.

In other aspects, a therapeutically effective amount of a stressor (e.g., ultrasonic radiation) reduces senescent cell burden in an individual by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 100%. In other aspects of this embodiment, a therapeutically effective amount of low frequency ultrasound (LFA) reduces the senescent cell burden by, e.g., at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, at most 95% or at most 100%. In yet other aspects of this embodiment, a therapeutically effective amount of a stressor (e.g., ultrasonic radiation) reduces senescent cell burden by, e.g., about 10% to about 100%, about 10% to about 90%, about 10% to about 80%, about 10% to about 70%, about 10% to about 60%, about 10% to about 50%, about 10% to about 40%, about 20% to about 100%, about 20% to about 90%, about 20% to about 80%, about 20% to about 20%, about 20% to about 60%, about 20% to about 50%, about 20% to about 40%, about 30% to about 100%, about 30% to about 90%, about 30% to about 80%, about 30% to about 70%, about 30% to about 60%, or about 30% to about 50%. In aspects, senescent cell burden is measured using biomarkers or comparing cell morphology. In aspects, the reduction in senescent cell burden slows/reverses signs of aging.

Certain embodiments of the present invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the present invention so claimed are inherently or expressly described and enabled herein.

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only, and is not intended to limit the scope of the present invention, which is defined solely by the claims. Accordingly, the present invention is not limited to that precisely as shown and described.

What is claimed is:

1. A method of treating an ailment in a subject, the method comprising administering a therapeutic amount of pulsed or modulated ultrasound to the subject.

2. The method of claim 1, wherein the ultrasound is low frequency ultrasound (LFU).

3. The method of claim 1, wherein the ultrasound is administered at 30-100 kHz.

4. The method of claim 1, wherein the ultrasound is applied to senescent cells.

5. The method of claim 1, wherein the ailment is a senescence-associated disease or disorder.

6. The method of claim 4, wherein the senescence-associated disease or disorder is one or more of atherosclerosis, diabetes, osteoarthritis, osteoporosis, hypertension, arthritis, cataracts, cancer, Alzheimer's disease, chronic obstructive pulmonary disease (COPD) and idiopathic pulmonary fibrosis, hair graying, sarcopenia, adiposity, neurogenesis, fibrosis and glaucoma.

7. The method of claim 1, wherein the ailment is a mitochondrial disease.

8. The method of claim 7, wherein the mitochondrial disease causes one or more of seizure, ataxia, cortical blandness, dystonia, exercise intolerance, ophthalmoplegia, optic atrophy, cataracts, diabetic mellitus, short stature, cardiomyopathy, sensorineural hearing loss, kidney failure, blindness, hearing loss, movement disorders (ataxia), dementia, cardiovascular disease, muscle weakness, renal dysfunction and endocrine disorders.

9. The method of claim 1, wherein the ultrasound is administered with a Rho kinase inhibitor or a senolytic agent.

10. The method of claim 9, wherein the senolytic agent is one or more of erastin, sulfasalazine, sorafenib, (1S, 3R)-RSL3, ML162, ML210, L-glutamic acid, L-buthionine sulfoximine, ML 210, simvastatin, sorafenib and sulfasalazine.

11. The method of claim 1, wherein the ultrasound is administered in one or more treatments, each treatment having a duration of 30 to 45 minutes.

12. A method of slowing the aging process and/or reducing signs of aging in a subject, the method comprising administering a therapeutic amount of pulsed or modulated ultrasound to the subject.

13. The method of claim 12, wherein the ultrasound is low frequency ultrasound (LFU).

14. The method of claim 12, wherein the ultrasound is administered with a Rho kinase inhibitor or a senolytic agent.

15. The method of claim 14, wherein the senolytic agent is one or more of erastin, sulfasalazine, sorafenib, (1S, 3R)-RSL3, ML162, ML210, L-glutamic acid, L-buthionine sulfoximine, ML 210, simvastatin, sorafenib and sulfasalazine.

16. The method of claim 12, wherein the ultrasound is administered in one or more treatments, each treatment having a duration of 30 to 45 minutes.

17. A method of treating an ailment associated with an elevated senescent cell burden, the method comprising:
a) identifying a tissue with an elevated senescent cell burden, and
b) administering pulses of low frequency ultrasound (LFU) to the tissue, thereby reducing the number of senescent cells in the tissue.

18. The method of claim 17, wherein the ailment associated with an elevated senescent cell burden is one or more of atherosclerosis, diabetes, osteoarthritis, osteoporosis, hypertension, arthritis, cataracts, cancer, Alzheimer's disease, chronic obstructive pulmonary disease (COPD) and idiopathic pulmonary fibrosis, hair graying, sarcopenia, adiposity, neurogenesis, fibrosis and glaucoma.

19. The method of claim 18, wherein the low frequency ultrasound is administered at pulses of about 30-100 kHz.

20. The method of claim 18, wherein the ultrasound (a) improves wound healing, (b) improves bone/tissue healing, (c) improve perfusion to tissue and/or (d) improves recovery from injury.
